SEQUENCE LISTING (73 sequences)

SEQ ID NO: 1 - protein, 16 aa, Artificial Sequence
  Description of Artificial Sequence Synthetic peptide
CYFDDSSNVLCKKYRS

SEQ ID NO: 2 - protein, 16 aa, Artificial Sequence
  Description of Artificial Sequence Synthetic peptide
CYFDDSSNVLCKKYRS

SEQ ID NO: 3 - protein, 16 aa, Artificial Sequence
  Description of Artificial Sequence Synthetic peptide
CYFDDSSNVLCKKYRS

SEQ ID NO: 4 - protein, 16 aa, Artificial Sequence
  Description of Artificial Sequence Synthetic peptide
CYFDDSSNVLCKKYRS

SEQ ID NO: 5 - protein, 16 aa, Artificial Sequence
  Description of Artificial Sequence Synthetic peptide
CYFDDSSNVLCKKYRS

SEQ ID NO: 6 - protein, 11 aa, Artificial Sequence
  Description of Artificial Sequence Synthetic peptide
CYFDDSSNVLC

SEQ ID NO: 7 - protein, 14 aa, Artificial Sequence
  Description of Artificial Sequence Synthetic peptide
CYFDDSSNVLCKKY

SEQ ID NO: 8 - protein, 12 aa, Artificial Sequence
  Description of Artificial Sequence Synthetic peptide
CYFDDSSNVLCK

SEQ ID NO: 9 - protein, 13 aa, Artificial Sequence
  Description of Artificial Sequence Synthetic peptide
CYFDDSSNVLCKK

SEQ ID NO: 10 - protein, 16 aa, Artificial Sequence
  Description of Artificial Sequence Synthetic peptide
CYFDDSSNVLCKRYRS

SEQ ID NO: 11 - protein, 16 aa, Artificial Sequence
  Description of Artificial Sequence Synthetic peptide
CYFDDSSNVLCKRYRS

SEQ ID NO: 12 - protein, 16 aa, Artificial Sequence
  Description of Artificial Sequence Synthetic peptide
CYYDDSSSVLCKKYRS

SEQ ID NO: 13 - protein, 16 aa, Artificial Sequence
  Description of Artificial Sequence Synthetic peptide
CYYDDSSSVLCKKYRS

SEQ ID NO: 14 - protein, 16 aa, Artificial Sequence
  Description of Artificial Sequence Synthetic peptide
CYYDNSSSVLCKKYRS

SEQ ID NO: 15 - protein, 16 aa, Artificial Sequence
  Description of Artificial Sequence Synthetic peptide
CYYDNSSSVLCKKYRS

SEQ ID NO: 16 - protein, 16 aa, Artificial Sequence
  Description of Artificial Sequence Synthetic peptide
CYYDNSSSVLCKRYRS

SEQ ID NO: 17 - protein, 16 aa, Artificial Sequence
  Description of Artificial Sequence Synthetic peptide
CYYDNSSSVLCKRYRS

SEQ ID NO: 18 - protein, 16 aa, Artificial Sequence
  Description of Artificial Sequence Synthetic peptide
CYYDNSSSVLCKRYRS

SEQ ID NO: 19 - protein, 16 aa, Artificial Sequence
  Description of Artificial Sequence Synthetic peptide
CXXXXXXXXXCXXXXX

SEQ ID NO: 20 - protein, 16 aa, Artificial Sequence
  Description of Artificial Sequence Synthetic peptide
CXXXXXXXXXCXXXXX

SEQ ID NO: 21 - protein, 16 aa, Artificial Sequence
  Description of Artificial Sequence Synthetic peptide
CXXXXXXXXXCXXYXX

SEQ ID NO: 22 - protein, 16 aa, Artificial Sequence
  Description of Artificial Sequence Synthetic peptide
CXXXXXXXXXCXXRXX

SEQ ID NO: 23 - protein, 16 aa, Artificial Sequence
  Description of Artificial Sequence Synthetic peptide
CXXXXXXXXXCXXXXX

SEQ ID NO: 24 - protein, 16 aa, Artificial Sequence
  Description of Artificial Sequence Synthetic peptide
CXXXXXXXXXCXKXXX

SEQ ID NO: 25 - protein, 16 aa, Artificial Sequence
  Description of Artificial Sequence Synthetic peptide
CYYFNSSQVLCKRYRS

SEQ ID NO: 26 - protein, 16 aa, Artificial Sequence
  Description of Artificial Sequence Synthetic peptide
CYFDDSSNVLCKKYRS

SEQ ID NO: 27 - protein, 16 aa, Artificial Sequence
  Description of Artificial Sequence Synthetic peptide
CYFDDSSNVLCKKARS

SEQ ID NO: 28 - protein, 16 aa, Artificial Sequence
  Description of Artificial Sequence Synthetic peptide
CYFDDSSNVLCKKARS

SEQ ID NO: 29 - protein, 16 aa, Artificial Sequence
  Description of Artificial Sequence Synthetic peptide
CYFDDSSNVLCKKHRS

SEQ ID NO: 30 - protein, 16 aa, Artificial Sequence
  Description of Artificial Sequence Synthetic peptide
CYFDDSSNVLCKKHRS

SEQ ID NO: 31 - protein, 16 aa, Artificial Sequence
  Description of Artificial Sequence Synthetic peptide
CYFDDSSNVLCKKYRS

SEQ ID NO: 32 - protein, 16 aa, Artificial Sequence
  Description of Artificial Sequence Synthetic peptide
CYFDDSSNVLCKKYRS

SEQ ID NO: 33 - protein, 16 aa, Artificial Sequence
  Description of Artificial Sequence Synthetic peptide
CYFDDSSNVLCKKYRS

SEQ ID NO: 34 - protein, 16 aa, Artificial Sequence
  Description of Artificial Sequence Synthetic peptide
CYFDDSSNVLCKKYRS

SEQ ID NO: 35 - protein, 16 aa, Artificial Sequence
  Description of Artificial Sequence Synthetic peptide
CYFNDSSQVLCKRYRS

SEQ ID NO: 36 - protein, 16 aa, Artificial Sequence
  Description of Artificial Sequence Synthetic peptide
CYFNDSSQVLCKRYRS

SEQ ID NO: 37 - protein, 16 aa, Artificial Sequence
  Description of Artificial Sequence Synthetic peptide
CYFDDNSNVLCRKYRS

SEQ ID NO: 38 - protein, 16 aa, Artificial Sequence
  Description of Artificial Sequence Synthetic peptide
CYFDDNSNVLCRKYRS

SEQ ID NO: 39 - protein, 16 aa, Artificial Sequence
  Description of Artificial Sequence Synthetic peptide
CYFDDSSNTLCKKYRS

SEQ ID NO: 40 - protein, 16 aa, Artificial Sequence
  Description of Artificial Sequence Synthetic peptide
CYFDDSSNTLCKKYRS

SEQ ID NO: 41 - protein, 16 aa, Artificial Sequence
  Description of Artificial Sequence Synthetic peptide
CYFDDSSNALCKKYRS

SEQ ID NO: 42 - protein, 16 aa, Artificial Sequence
  Description of Artificial Sequence Synthetic peptide
CYFDDSSNALCKKYRS

SEQ ID NO: 43 - protein, 16 aa, Artificial Sequence
  Description of Artificial Sequence Synthetic peptide
CYYFSSSFVLCKRYRS

SEQ ID NO: 44 - protein, 16 aa, Artificial Sequence
  Description of Artificial Sequence Synthetic peptide
CYYFSSSFVLCKRYRS

SEQ ID NO: 45 - protein, 16 aa, Artificial Sequence
  Description of Artificial Sequence Synthetic peptide
CYFFDSSNVLCKKYRS

SEQ ID NO: 46 - protein, 16 aa, Artificial Sequence
  Description of Artificial Sequence Synthetic peptide
CYFFDSSNVLCKKYRS

SEQ ID NO: 47 - protein, 16 aa, Artificial Sequence
  Description of Artificial Sequence Synthetic peptide
CYFXDSSNVLCKKYRS

SEQ ID NO: 48 - protein, 16 aa, Artificial Sequence
  Description of Artificial Sequence Synthetic peptide
CYFXDSSNVLCKKYRS

SEQ ID NO: 49 - protein, 16 aa, Artificial Sequence
  Description of Artificial Sequence Synthetic peptide
CYFDDFSNVLCKKYRS

SEQ ID NO: 50 - protein, 16 aa, Artificial Sequence
  Description of Artificial Sequence Synthetic peptide
CYFDDFSNVLCKKYRS

SEQ ID NO: 51 - protein, 16 aa, Artificial Sequence
  Description of Artificial Sequence Synthetic peptide
CYFDDSFNVLCKKYRS

SEQ ID NO: 52 - protein, 16 aa, Artificial Sequence
  Description of Artificial Sequence Synthetic peptide
CYFDDSFNVLCKKYRS

SEQ ID NO: 53 - protein, 16 aa, Artificial Sequence
  Description of Artificial Sequence Synthetic peptide
CYFDDSSFVLCKKYRS

SEQ ID NO: 54 - protein, 16 aa, Artificial Sequence
  Description of Artificial Sequence Synthetic peptide
CYFDDSSFVLCKKYRS

SEQ ID NO: 55 - protein, 16 aa, Artificial Sequence
  Description of Artificial Sequence Synthetic peptide
CYYFNSSSVLCKRYRS

SEQ ID NO: 56 - protein, 16 aa, Artificial Sequence
  Description of Artificial Sequence Synthetic peptide
CYYFNSSSVLCKRYRS

SEQ ID NO: 57 - protein, 16 aa, Artificial Sequence
  Description of Artificial Sequence Synthetic peptide
CYFFNSSQVLCKRYRS

SEQ ID NO: 58 - protein, 16 aa, Artificial Sequence
  Description of Artificial Sequence Synthetic peptide
CYFFNSSQVLCKRYRS

SEQ ID NO: 59 - protein, 16 aa, Artificial Sequence
  Description of Artificial Sequence Synthetic peptide
CYFYNSSQVLCKRYRS

SEQ ID NO: 60 - protein, 16 aa, Artificial Sequence
  Description of Artificial Sequence Synthetic peptide
CYFYNSSQVLCKRYRS

SEQ ID NO: 61 - protein, 16 aa, Artificial Sequence
  Description of Artificial Sequence Synthetic peptide
CYYFNSSQDLCKRYRS

SEQ ID NO: 62 - protein, 16 aa, Artificial Sequence
  Description of Artificial Sequence Synthetic peptide
CYYFNSSQDLCKRYRS

SEQ ID NO: 63 - protein, 16 aa, Artificial Sequence
  Description of Artificial Sequence Synthetic peptide
CYYFNSSQSLCKRYRS

SEQ ID NO: 64 - protein, 16 aa, Artificial Sequence
  Description of Artificial Sequence Synthetic peptide
CYYFNSSQSLCKRYRS

SEQ ID NO: 65 - protein, 16 aa, Artificial Sequence
  Description of Artificial Sequence Synthetic peptide
CYFDFSSNVLCKKYRS

SEQ ID NO: 66 - protein, 16 aa, Artificial Sequence
  Description of Artificial Sequence Synthetic peptide
CYFDFSSNVLCKKYRS

SEQ ID NO: 67 - protein, 16 aa, Artificial Sequence
  Description of Artificial Sequence Synthetic peptide
CYYFNSSQVLCKRYRS

SEQ ID NO: 68 - protein, 15 aa, Artificial Sequence
  Description of Artificial Sequence Synthetic peptide
YYFSSSFVLCKRYRS

SEQ ID NO: 69 - protein, 16 aa, Artificial Sequence
  Description of Artificial Sequence Synthetic peptide
CXXXXXXXXXCXXHXX

SEQ ID NO: 70 - protein, 16 aa, Artificial Sequence
  Description of Artificial Sequence Synthetic peptide
CXXXXXXXXXCXXHXX

SEQ ID NO: 71 - protein, 16 aa, Artificial Sequence
  Description of Artificial Sequence Synthetic peptide
CXXXXXXXXXCXXAXX

SEQ ID NO: 72 - protein, 16 aa, Artificial Sequence
  Description of Artificial Sequence Synthetic peptide
CXXXXXXXXXCXXAXX

SEQ ID NO: 73 - protein, 16 aa, Artificial Sequence
  Description of Artificial Sequence Synthetic peptide
XYYFSSSFVLCKRYRS

(19) United States
(12) Patent Application Publication
(10) Pub. No.: US 2016/0137714 A1
(43) Pub. Date: May 19, 2016

(54) PEPTIDES WITH ENHANCED STABILITY AND THEIR USE IN METHODS FOR TREATING DISEASES

(71) Applicant: Thrasos Innovation, Inc., Montreal (CA)

(72) Inventors: Peter C. KECK, Millbury, MA (US); Philippe BEY, Boston, MA (US); Roger LEGER, Saint-Lambert (CA); Paul DROGARIS, Westmount (CA)

(21) Appl. No.: 14/938,964

(22) Filed: Nov. 12, 2015

Related U.S. Application Data
(60) Provisional application No. 62/180,230, filed on Jun. 16, 2015.
(60) Provisional application No. 62/130,882, filed on Mar. 10, 2015.
(60) Provisional application No. 62/128,820, filed on Mar. 5, 2015.
(60) Provisional application No. 62/078,693, filed on Nov. 12, 2014.

(51) Int. Cl. C07K 14/705

(52) U.S. Cl. CPC C07K 14/705; A61K 38/00

(57) ABSTRACT

The invention provides peptides that are useful for treating or preventing, or preventing the progression of a disorder associated with aberrant TDF polypeptide or TDFRP compound target molecule expression or activity, for example a kidney disease or disorder, in a subject. The invention also provides methods of treating a subject having a disorder associated with aberrant TDF polypeptide or TDFRP compound target molecule expression or activity, such as a kidney disease or disorder, comprising administering to the subject an effective amount of the peptides of the invention.

### RELATED APPLICATIONS

The instant application claims priority to U.S. Provisional Application No. 62/078,693, filed on Nov. 12, 2014; U.S. Provisional Application No. 62/128,820, filed on Mar. 5, 2015; U.S. Provisional Application No. 62/130,882, filed on Mar. 10, 2015; and U.S. Provisional Application No. 62/180,230, filed on Jun. 16, 2015. The entire contents of each of the foregoing applications are expressly incorporated herein by reference.

### SEQUENCE LISTING

The instant application contains a Sequence Listing which has been submitted electronically in ASCII format and is hereby incorporated by reference in its entirety. Said ASCII copy, created on Jan. 25, 2016, is named 119562-02205_SL.txt and is 21,700 bytes in size.

### BACKGROUND OF THE INVENTION

Cell differentiation is the central characteristic of tissue morphogenesis, which initiates during embryogenesis, and continues to various degrees throughout the life of an organism in adult tissue repair and regeneration mechanisms. The degree of morphogenesis in adult tissue varies among different tissues and is related, among other things, to the degree of cell turnover in a given tissue.

A number of different factors have been isolated in recent years, which appear to play a role in cell differentiation. It is anticipated that discovery of such factors which control cell differentiation and tissue morphogenesis will advance significantly the ability to repair and regenerate diseased, injured or damaged mammalian tissues and organs. Particularly useful areas for therapeutics include reconstructive surgery, the treatment of tissue degenerative diseases including, for example, arthritis, emphysema, osteoporosis, cardiomyopathy, cirrhosis, degenerative nerve diseases, inflammatory diseases, and cancer, and in the protection and/or regeneration of tissues, organs and limbs. The terms “morphogenetic” and “morphogenic” are often used interchangeably.

Recently, a distinct subfamily of the “superfamily” of structurally related polypeptides referred to in the art as the “Transforming Growth Factor-beta (TGF-beta; TGF-β) superfamily of polypeptides” have been identified as tissue morphogens. The members of this distinct “subfamily” of tissue morphogenic polypeptides share substantial amino acid sequence homology within their morphogenetically active C-terminal domains, including a conserved six or seven cysteine skeleton, and share the in vivo activity of inducing tissue-specific morphogenesis in a variety of organs and tissues. The polypeptides apparently contact and interact with progenitor cells e.g., by binding suitable cell surface molecules, predisposing or otherwise stimulating the cells to proliferate and differentiate in a morphogenetically permissive environment. Recent studies on cell surface receptor binding of various members of the TGF-beta polypeptide superfamily suggests that the peptides mediate their activity by interaction with two different receptors, referred to as Type I and Type II receptors. The Type I or Type II receptors are both serine/threonine kinases, and share similar structures: an intracellular domain that consists essentially of the kinase, a short, extended hydrophobic sequence sufficient to span the membrane one time, and an extracellular domain characterized by a high concentration of conserved cysteines.

Morphogenic polypeptides are capable of inducing the developmental cascade of cellular and molecular events that culminate in the formation of new organ-specific tissue, including any vascularization, connective tissue formation, and nerve innervation as required by the naturally occurring tissue. The polypeptides have been shown to induce morphogenesis of cartilage and bone, as well as, periodontal tissues, dentin, liver, heart, kidney and neural tissue, including retinal tissue. These tissue morphogenic polypeptides are recognized in the art as a distinct subfamily of polypeptides different from other members of the TGF-beta superfamily in that they share a high degree of sequence identity in the C-terminal domain and in that the tissue morphogenic polypeptides are able to induce, on their own, the full cascade of events that result in formation of functional tissue rather than merely inducing formation of fibrotic (scar) tissue. Specifically, members of the family of morphogenic polypeptides are capable of all of the following in a morphogenetically permissive environment: stimulating cell proliferation and cell differentiation, and supporting the growth and maintenance of differentiated cells. The morphogenic polypeptides also may act as endocrine, paracrine or autocrine factors. As a result of their biological activities, significant effort has been directed toward the development of morphogen-based therapeutics for treating injured or diseased mammalian tissue.

Nevertheless, there remains a need in the art for morphogen-based therapeutics for use in both prophylactic and therapeutic methods of treating a subject at risk of (or susceptible to) a disorder or having a disorder associated with aberrant Tissue Differentiation Factor (TDF) polypeptide or tissue differentiation factor related polypeptide (TDFRP) compound target molecule expression or activity.

### SUMMARY OF THE INVENTION

The present invention is based, in part, on the discovery by the inventors that the stability of a compound toward proteolytic cleavage could be enhanced by incorporating a D-amino acid or a N-Methyl amino acid into the peptide structure. Accordingly, the present invention relates to the design, preparation, and use of peptides or polypeptides (i.e., interchangeably which may be referred to as “compounds,” “peptides,” or “polypeptides” of the invention) for treating, inhibiting, reversing, and/or eliminating disorders associated with aberrant TDF polypeptide or TDFRP compound target molecule expression or activity including, for example kidney diseases or disorders.

In a first aspect, the present invention features a method of treating a subject having a disease or disorder associated with aberrant TDF polypeptide or TDFRP compound target molecule expression or activity, or a method of delaying the progression of a disorder associated with aberrant TDF polypeptide or TDFRP compound target molecule expression or activity, comprising administering to the subject an effective amount of a peptide comprising one or more of SEQ ID NO:1-72, thereby treating the disorder associated with aberrant TDF polypeptide or TDFRP compound target molecule expression or activity, or preventing the progression of the disorder associated with aberrant TDF polypeptide or TDFRP compound target molecule expression or activity, in the subject.

In one embodiment, the disease or disorder is a tissue degenerative disease. In a further embodiment, the tissue degenerative disease is selected from the group consisting of renal disease, heart disease, traumatic brain injury, stroke, atherosclerosis, arthritis, emphysema, osteoporosis, cardiomyopathy, cirrhosis, degenerative nerve disease, Holt-Oram disease, congenital disease, pulmonary disease, eye disease, diabetic nephropathy, degenerative bone disease, bone disorders, periodontal disease, chronic kidney disease, diabetes, cardiovascular disease, inflammatory disease, immune disease, skeletal disease, reproductive disease, hematopoetic disease, healing disorders, and cancer.

In another embodiment, the disease or disorder is treated by the regeneration of tissues, organs or limbs. In a further embodiment, the tissue is selected from the group consisting of: muscle, bone, skin, epithelial, heart, nerve, endocrine, vessel, cartilage, periodontal, liver, retinal, and connective tissue. In one embodiment, an effective amount of a peptide comprising one or more of SEQ ID NO:1-72 is useful to induce regenerative healing of bone defects or to preserve or restore healthy metabolic properties in diseased tissue. In a further embodiment, the bone defect is a fracture. In another embodiment, the diseased tissue is osteopenic bone tissue.

In another aspect, the present invention provides peptides for treating a kidney disease or disorder, comprising the amino acid sequence set forth as any one of SEQ ID NOs: 1-72. In one embodiment, the peptide has at least 70% identity to SEQ ID NO:1-72. In one embodiment, the peptide consists of the amino acid sequence set forth as any one of SEQ ID NOs:1-72. In one embodiment, the peptide has at least 70% identity to SEQ ID NO:25. In another embodiment, the peptide consists of the amino acid sequence of SEQ ID NO:25.

In another embodiment, the invention provides pharmaceutical compositions comprising one or more of the peptides described herein and a pharmaceutically acceptable carrier.

In another embodiment, the invention provides kits comprising one or more of the peptides described herein and instructions for use.

In another aspect, the invention provides methods of treating a subject having a kidney disease or disorder or a method of delaying the progression of a kidney disease or disorder in a subject, comprising administering to the subject an effective amount of a one or more peptides comprising SEQ ID NO:1-72, thereby treating the kidney disease or disorder, or preventing the progression of the kidney disease or disorder, in the subject. In one embodiment, the peptide comprises SEQ ID NO:25. In another embodiment, the peptide consists of SEQ ID NO:25.

In one embodiment, the peptide has at least 70% identity to a peptide comprising a peptide set forth as SEQ ID NO:1-72. In another embodiment, the peptide has at least 80% identity to a peptide comprising a peptide set forth as SEQ ID NO:1-72. In another embodiment, the peptide has at least 90% identity to a peptide comprising a peptide set forth as SEQ ID NO:1-72. In another embodiment, the peptide has at least 91%, 92%, 93%, 94%, 95%, 96%, 97%, 98%, or 99% identity to a peptide comprising a peptide set forth as SEQ ID NO:1-72. In one embodiment, the peptide has at least 70% identity to a peptide comprising a peptide set forth as SEQ ID NO:25. In another embodiment, the peptide has at least 80% identity to a peptide comprising a peptide set forth as SEQ ID NO:25. In another embodiment, the peptide has at least 90% identity to a peptide comprising a peptide set forth as SEQ ID NO:25. In another embodiment, the peptide has at least 91%, 92%, 93%, 94%, 95%, 96%, 97%, 98%, or 99% identity to a peptide comprising a peptide set forth as SEQ ID NO:25.

In another embodiment, the peptide consists of the amino acid sequence set forth as any one of SEQ ID NOs:1-72. In another embodiment, the peptide consists of SEQ ID NO:25.

In one embodiment, the peptide is formulated with a pharmaceutically acceptable carrier.

In one embodiment, the peptide is administered to the subject orally. In another embodiment, the peptide is administered to the subject topically, enterally, or parenterally.

In one embodiment, the disease or disorder is chronic kidney disease. In another embodiment, the disease or disorder is a renal dysfunction. In related embodiments, the renal dysfunction is selected from the group consisting of ureteral obstruction, acute and chronic renal failure, renal fibrosis, and diabetic nephropathy.

In one embodiment, a dosage of 0.0001 to 10,000 mg/kg body weight is administered to the subject per day. In another embodiment, the administered dosage is from 1 to 100 mg/kg body weight per day.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 is a graph that shows the pharmacokinetic profile of THR-123 in mouse kidney tissue.

FIG. 2 is a graph that shows the relative amount of metabolites of THR-123 in rat kidney tissue 10 minutes post IV administration.

FIG. 3 is a graph that shows in vitro rat plasma stability of compounds I to IV at 37° C.

FIG. 4 is a graph that shows rat plasma proteolysis of stabilized peptides.

FIG. 5 is a graph that shows plasma metabolite profile after administration of THR-123 s.c. (1 mg/kg).

FIG. 6 is a graph that shows plasma metabolite profile after IV administration of 10 mg/kg THR-123.

FIG. 7 is a graph that shows plasma metabolite profile after s.c. administration of 1 mg/kg Compound III.

FIG. 8 is a graph that shows the pharmacokinetics profile of Compound III after IV administration.

FIG. 9 is a graph depicting the first 240 minutes of the time-course and disappearance of the peptides THR-123 and GPR-405 in heparin-treated fresh rat plasma.

FIG. 10 is a graph depicting the first 1440 minutes of the time-course and disappearance of the peptides THR-123 and GPR-405 in heparin-treated fresh rat plasma.

FIG. 11 is a graph depicting the area under the curve (AUC) between time 0 and 240 minutes for THR-123 and GPR-405.

FIG. 12 is a graph depicting the half-life for THR-123 and GPR-405.

FIG. 13 is a graph that shows results of the pharmacokinetic (PK) studies.

FIGS. 14A and 14B show mouse plasma PK profile after subcutaneous (S.C.) administration.

FIG. 15 is a graph depicting the log-linear regression fit for GPR-405 of the last 3 time points (30, 60, and 240 minutes) that was used to estimate the terminal decay rate (K′).

FIG. 16 is a graph depicting the overall PK profile of GPR-405, with each point representing the mean±SD of the plasma concentration (n=2-3 mice per point).

FIG. 17 is micrographs of H&E stained 6 μl rate kidney sections at 10× magnification 21 days post ADR administration, treated daily with either vehicle, enalapril, or GPR-405.

FIG. 18 is a graph that shows the effect of GPR-405 and enalapril on the time-course of the development of proteinuria.

FIG. 19 is a graph that shows the effect of various Compounds on proteinuria 15 days post-adriamycin treatment.

FIG. 20 is a graph that shows the effect of various Compounds on the area under the curve, days 0-15 (AUC_{d0-15}).

FIG. 21 is a graph that shows the effect of various Compounds on ischemia-reperfusion injury model (IRI)-induced rise in serum creatinine at 24 hours.

FIG. 22 is a graph showing the development of Adriamycine (ADR)-induced proteinuria over time, and the effects of vehicle (Veh), enalapril (Enal) or GPR-405 treatment.

FIG. 23 is a graph showing the development of ADR-induced proteinuria over time, and the effects of vehicle (Veh), enalapril (Enal) or GPR-405 treatment as represented by AUC_{d0-21}.

FIG. 24 is a graph depicting the effect of treatment with vehicle, enalapril, or GPR-405 on changes in serum creatine (sCr) and blood urea nitrogen (BUN) following ADR treatment.

FIG. 25 is a graph depicting the effects of daily vehicle administration of mRNA levels of 19 genes in dissected kidney cortices of rats 21 days post ADR.

FIG. 26 is a graph depicting the effects of daily enalapril administration (50 mg/kg) of mRNA levels of 19 genes in dissected kidney cortices of rats 21 days post ADR.

FIG. 27 is a graph depicting the effects of daily GPR-405 administration (300 nmol/kg) of mRNA levels of 19 genes in dissected kidney cortices of rats 21 days post ADR.

FIG. 28 is a graph depicting the effects of daily vehicle, enalapril, and GPR-405 administration of mRNA levels of six genes in dissected kidney cortices of rats 21 days post ADR.

FIG. 29 is a series of graphs depicting the correlation between proteinuria levels and the expression levels of four genes (Kim1, osteopontin, NGAL, and clusterin) in rats induced with ADR-induced protein urea at day 1 and administered either enalapril or GPR-405.

FIG. 30 is a micrograph of H&E stained rat kidney sections 21 days post ADR insult treated daily with either vehicle, enalapril, or GPR-405.

FIG. 31 is a Table that shows the results of peptide stability studies. Peptide to internal standard peak area ratios were used calculate percentage remaining peptide at 60 minutes and 120 minutes. Peptides are identified by GPR number (GPR#) and SEQ ID NO.

### DETAILED DESCRIPTION OF THE INVENTION

The present invention is based, in part, on the discovery by the inventors that the stability of a compound toward proteolytic cleavage could be enhanced by incorporating a D-amino acid or N-Methyl amino acid into the peptide structure. The peptides of the invention, and pharmaceutical compositions comprising these peptides, are useful in methods of treating a subject having a disease or disorder associated with aberrant TDF polypeptide or TDFRP compound target molecule expression or activity, or a method of delaying the progression of a disorder associated with aberrant TDF polypeptide or TDFRP compound target molecule expression or activity. For example, the disease or disorder treated by the peptides of the invention may be a tissue degenerative disease, for example, but not limited to, renal disease, heart disease, traumatic brain injury, stroke, atherosclerosis, arthritis, emphysema, osteoporosis, cardiomyopathy, cirrhosis, degenerative nerve disease, Holt-Oram disease, congenital disease, pulmonary disease, eye disease, diabetic nephropathy, degenerative bone disease, bone disorders, periodontal disease, chronic kidney disease, diabetes, cardiovascular disease, inflammatory disease, immune disease, skeletal disease, reproductive disease, hematopoetic disease, healing disorders, and cancer.

In certain embodiments, the disease or disorder is treated by the regeneration of tissues, organs or limbs, for example TDFRP-based therapeutic compositions are useful to induce regenerative healing of bone defects such as fractures, as well as, to preserve or restoring healthy metabolic properties in diseased tissue, e.g., osteopenic bone tissue.

In particular embodiments, the peptides of the invention (as further described herein) are useful for treating, inhibiting, reversing, and/or eliminating kidney diseases or disorders.

### DEFINITIONS

The present invention may be understood more readily by reference to the following detailed description of embodiments of the invention and the Examples included therein. Before the present methods and techniques are disclosed and described, it is to be understood that this invention is not limited to specific analytical or synthetic methods as such may, of course, vary. It is also to be understood that the terminology used herein is for the purpose of describing particular embodiments only and is not intended to be limiting. Unless defined otherwise, all technical and scientific terms used herein have the meaning commonly understood by one of ordinary skill in the art to which this invention belongs.

As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

“D-amino acid,” as used herein, refers to a particular isomeric form of an amino acid. D-amino acids are mirror images of L-amino acids, where the chirality at carbon alpha has been inverted.

“Aromatic amino acid,” as used herein, refers to a hydrophobic amino acid having a side chain containing at least one ring having a conjugated electron system (aromatic group). The aromatic group may be further substituted with substituent groups such as alkyl, alkenyl, alkynyl, hydroxyl, sulfanyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chlorophenylalanine, 2-fluorophenylalanine, 3-fluorophenylalanine and 4-fluorophenylalanine.

“Aliphatic amino acid,” as used herein, refers to an apolar amino acid having a saturated or unsaturated straight chain, branched or cyclic hydrocarbon side chain. Examples of genetically encoded aliphatic amino acids include alanine, leucine, valine and isoleucine. Examples of non-encoded aliphatic amino acids include norleucine (Nle).

“Acidic amino acid,” as used herein, refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic amino acid,” as used herein, refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Polar amino acid,” as used herein, refers to a hydrophilic amino acid having a side chain that is uncharged at physiological pH, but which has a bond in which the pair of electrons shared in common by two atoms is held more closely by one of the atoms. Examples of genetically encoded polar amino acids include asparagine and glutamine. Examples of non-genetically encoded polar amino acids include citrulline, N-acetyl lysine and methionine sulfoxide.

As will be appreciated by those having skill in the art, the above classifications are not absolute—several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories.

As used herein, the term “disease or disorder associated with aberrant TDF polypeptide or TDFRP compound target expression or activity” is meant to refer to any condition in which Tissue Differentiation Factor (TDF) polypeptide or tissue differentiation factor related polypeptide (TDFRP) target (such as TDF receptors) expression or activity is increased, decreased, or abnormal compared to a normal control. The term encompasses any condition that would benefit from treatment with the formulations of the invention, e.g., a disorder requiring treatment with the peptides of the invention (e.g., SEQ ID NOs 1-72). This includes chronic and acute disorders or diseases including those pathological conditions that predispose the subject to the disorder in question. For example, the disease or disorder may be a tissue degenerative disease, for example, but not limited to, renal disease, heart disease, traumatic brain injury, stroke, atherosclerosis, arthritis, emphysema, osteoporosis, cardiomyopathy, cirrhosis, degenerative nerve disease, Holt-Oram disease, congenital disease, pulmonary disease, eye disease, diabetic nephropathy, degenerative bone disease, bone disorders, periodontal disease, chronic kidney disease, diabetes, cardiovascular disease, inflammatory disease, immune disease, skeletal disease, reproductive disease, hematopoetic disease, healing disorders, and cancer.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of a compound, as used herein, is a quantity sufficient to achieve a desired therapeutic and/or prophylactic effect, for example, an amount which results in the prevention of or a decrease in the symptoms associated with a disease that is being treated, e.g., a kidney disease or disorder. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day, to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

An “isolated” or “purified” polypeptide or polypeptide or biologically-active portion thereof is substantially free of cellular material or other contaminating polypeptides from the cell or tissue source from which the tissue differentiation factor-related polypeptide is derived, or substantially free from chemical precursors or other chemicals when chemically synthesized.

The term “variant,” as used herein, refers to a compound that differs from the compound of the present invention, but retains essential properties thereof. A non-limiting example of this is a polynucleotide or polypeptide compound having conservative substitutions with respect to the reference compound, commonly known as degenerate variants. Another non-limiting example of a variant is a compound that is structurally different, but retains the same active domain of the compounds of the present invention. Variants include N-terminal or C-terminal extensions, capped amino acids, modifications of reactive amino acid side chain functional groups, e.g., branching from lysine residues, pegylation, and/or truncations of a polypeptide compound. Generally, variants are overall closely similar, and in many regions, identical to the compounds of the present invention. Accordingly, the variants may contain alterations in the coding regions, non-coding regions, or both.

As used herein, the term “local” or “locally,” as in local administration or co-administration of one or more therapeutics, refers to the delivery of a therapeutic agent to a bodily site that is proximate or nearby the site of an injury, adjacent or immediately nearby the site of an injury, at the perimeter of or in contact with an injury site, or within or inside the injured tissue or organ. Local administration generally excludes systemic administration routes.

As used herein, the term “pharmaceutically effective regimen” refers to a systematic plan for the administration of one or more therapeutic agents which includes aspects such as drug concentrations, amounts or levels, timing, and repetition, and any changes therein made during the course of the drug administration, which when administered is effective in treating a kidney disease or disorder. The skilled artisan, which will generally include practicing physicians who are treating patients having a fibrotic condition, will appreciate and understand how to determine a pharmaceutically effective regimen without undue experimentation.

As used herein, the term “co-administering,” or “co-administration,” and the like refers to the act of administering two or more agents, therapeutics, compounds, therapies, or the like, at or about the same time. The order or sequence of administering the different agents of the invention may vary and is not confined to any particular sequence. Co-administering may also refer to the situation where two or more agents are administered to different regions of the body or via different delivery schemes, e.g., where a first agent is administered systemically and a second agent is administered locally at the site of tissue injury, or where a first agent is administered locally and a second agent is administering systemically into the blood.

The term “pharmaceutically acceptable” as used herein, refers to a material, (e.g., a carrier or diluent), which does not abrogate the biological activity or properties of the compounds described herein, and is relatively nontoxic (i.e., the material is administered to an individual without causing undesirable biological effects or interacting in a deleterious manner with any of the components of the composition in which it is contained).

As used herein, the term “selectively” means tending to occur at a higher frequency in one population than in another population.

As used herein, the term “coupled,” as in reference to two or more agents being “coupled” together, refers to a covalent or otherwise stable association between the two or more agents. For example, a therapeutic peptide of the invention may be coupled with a second agent via a covalent bond, a covalently tethered linker moiety, or through ionic interactions. Preferably, the one or more agents that are coupled together retain substantial their same independent functions and characteristics. For example, the therapeutic agent when coupled to another agent may retain its same activity as if it were independent.

As used herein, the term “regimen” refers to the various parameters that characterize how a drug or agent is administered, including, the dosage level, timing, and iterations, as well as the ratio of different drugs or agents to one another. The term “pharmaceutically effective regimen” refers to a particular regimen which provides a desired therapeutic result or effect, including treating, inhibiting, reversing, and/or eliminating kidney diseases or disorders. The term “iterations” refer to the general concept of repeating sets of administering one or more agents. For example, a combination of drug X and drug Y may be given (co-administered at or about at the same time and in any order) to a patient on a first day at dose Z. Drugs X and Y may then be administered (co-administered at or about at the same time and in any order) again at dose Z, or another dose, on a second day. The timing between the first and second days can be 1 day or anywhere up to several days, or a week, or several weeks, or months. The iterative administrations may also occur on the same day, separated by a specified number of minutes (e.g., 10 minutes, 20 minutes, 30 minutes or more) or hours (e.g., 1 hour, 2 hours, 4 hours, 6 hours, 12 hours). An effective dosing regimen may be determinable by those of ordinary skill in the art, e.g., prescribing physician, using standard practices.

Peptides of the Invention

The present invention provides peptides, and pharmaceutical compositions comprising these peptides, for the use in methods of treating a subject having a disease or disorder associated with aberrant TDF polypeptide or TDFRP compound target molecule expression or activity, or a method of delaying the progression of a disorder associated with aberrant TDF polypeptide or TDFRP compound target molecule expression or activity.

In one aspect, the present invention provides peptides, and pharmaceutical compositions comprising these peptides, for the use in treating, inhibiting, reversing, and/or eliminating kidney diseases or disorders.

Variants, analogs, homologs, or fragments of these peptides, such as species homologs, are also included in the present invention, as well as degenerate forms thereof. The peptides of the present invention may be capped on the N-terminus or the C-terminus or on both the N-terminus and the C-terminus. The peptides of the present invention may be pegylated, or modified, e.g., branching, at any amino acid residue containing a reactive side chain, e.g., lysine residue. The peptides of the present invention may be linear or cyclized or otherwise constrained. The tail sequence of the peptide may vary in length.

The peptides can contain natural amino acids, non-natural amino acids, D-amino acids and L-amino acids, N-Methyl (alkyl) amino acids and any combinations thereof. In certain embodiments, the compounds of the invention can include commonly encountered amino acids, which are not genetically encoded. These non-genetically encoded amino acids include, but are not limited to, β-alanine (β-Ala) and other omega-amino acids such as 3-aminopropionic acid (Dap), 2,3-diaminopropionic acid (Dpr), 4-aminobutyric acid and so forth; alpha-aminoisobutyric acid (Aib); epsilon-aminohexanoic acid (Aha); delta-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-C1)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); beta-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH.sub.2)); N-methyl valine (MeVal); homocysteine (hCys) and homoserine (hSer).

In preferred embodiments, the peptides of the invention have the general structure shown in THR-123 (SEQ ID NO: 1), comprising a deletion or one or more substitutions. In further preferred embodiments of the invention, the peptides contain a substitution of one or more D-amino acids, or N-Methyl amino acids, or an N-alkyl amino acids.

In further preferred embodiments, the amino acid residues of SEQ ID NOs:1-72, analogs or homologs of SEQ ID NOs:1-72 include genetically-encoded L-amino acids, naturally occurring non-genetically encoded L-amino acids, synthetic D-amino acids, or D-enantiomers of all of the above.

Representative peptides according to exemplary embodiments of the present invention are summarized in Table 1.

TABLE 1

| SEQ ID | |
|---|---|
| NO | Sequence |
| 1 | Cys-Tyr-Phe-Asp-Asp-Ser-Ser-Asn-Val-Leu- |
| | Cys-Lys-Lys-Tyr-Arg-Ser |
| 2 | Cys-Tyr-Phe-Asp-Asp-Ser-Ser-Asn-Val-Leu- |
| | Cys-Lys-(D)lys-Tyr-Arg-Ser |
| 3 | Cys-Tyr-Phe-Asp-Asp-Ser-Ser-Asn-Val-Leu- |
| | Cys-Lys-Lys-(D)tyr-Arg-Ser |
| 4 | Cys-Tyr-Phe-Asp-Asp-Ser-Ser-Asn-Val-Leu- |
| | Cys-Lys-Lys-Tyr-(D)arg-Ser |
| 5 | Cys-Tyr-Phe-Asp-Asp-Ser-Ser-Asn-Val-Leu- |
| | Cys-Lys-Lys-Tyr-(Nα-Me)Arg-Ser |
| 6 | Cys-Tyr-Phe-Asp-Asp-Ser-Ser-Asn-Val-Leu- |
| | Cys |
| 7 | Cys-Tyr-Phe-Asp-Asp-Ser-Ser-Asn-Val-Leu- |
| | Cys-Lys-Lys-Tyr |
| 8 | Cys-Tyr-Phe-Asp-Asp-Ser-Ser-Asn-Val-Leu- |
| | Cys-Lys |
| 9 | Cys-Tyr-Phe-Asp-Asp-Ser-Ser-Asn-Val-Leu- |
| | Cys-Lys-Lys |
| 10 | Cys-Tyr-Phe-Asp-Asp-Ser-Ser-Asn-Val-Leu- |
| | Cys-Lys-Arg-Tyr-Arg-Ser |
| 11 | Cys-Tyr-Phe-Asp-Asp-Ser-Ser-Asn-Val-Leu- |
| | Cys-Lys-Arg-(D)tyr-Arg-Ser |
| 12 | Cys-Tyr-Tyr-Asp-Asp-Ser-Ser-Ser-Val-Leu- |
| | Cys-Lys-Lys-Tyr-Arg-Ser |
| 13 | Cys-Tyr-Tyr-Asp-Asp-Ser-Ser-Ser-Val-Leu- |
| | Cys-Lys-Lys-(D)tyr-Arg-Ser |
| 14 | Cys-Tyr-Tyr-Asp-Asn-Ser-Ser-Ser-Val-Leu- |
| | Cys-Lys-Lys-Tyr-Arg-Ser |
| 15 | Cys-Tyr-Tyr-Asp-Asn-Ser-Ser-Ser-Val-Leu- |
| | Cys-Lys-Lys-(D)tyr-Arg-Ser |
| 16 | Cys-Tyr-Tyr-Asp-Asn-Ser-Ser-Ser-Val-Leu- |
| | Cys-Lys-Arg-Tyr-Arg-Ser |
| 17 | Cys-Tyr-Tyr-Asp-Asn-Ser-Ser-Ser-Val-Leu- |
| | Cys-Lys-Arg-(D)tyr-Arg-Ser |
| 18 | Cys-Tyr-Tyr-Asp-Asn-Ser-Ser-Ser-Val-Leu- |
| | Cys-Lys-Arg-Tyr-(Nα-Me)Arg-Ser |
| 25 | Cys-Tyr-Tyr-Phe-Asn-Ser-Ser-Gln-Val-Leu- |
| | Cys-Lys-Arg-(D)Tyr-Arg-Ser |
| 19 | CX₂X₃X₄X₅X₆X₇X₈X₉X₁₀CX₁₂X₁₃X₁₄X₁₅X₁₆ |
| 20 | CX₂X₃X₄X₅X₆X₇X₈X₉X₁₀CX₁₂X₁₃(D)X₁₄X₁₅X₁₆ |
| 21 | CX₂X₃X₄X₅X₆X₇X₈X₉X₁₀CX₁₂X₁₃(D)TyrX₁₅X₁₆ |
| 22 | CX₂X₃X₄X₅X₆X₇X₈X₉X₁₀CX₁₂X₁₃(Nα-Me)ArgX₁₅X₁₆ |
| 23 | CX₂X₃X₄X₅X₆X₇X₈X₉X₁₀CX₁₂X₁₃X₁₄X₁₅X₁₆ |
| 24 | CX₂X₃X₄X₅X₆X₇X₈X₉X₁₀CX₁₂(D)lysX₁₄X₁₅X₁₆ |
| 26 | Cys-Tyr-Phe-Asp-Asp-Ser-Ser-Asn-Val-Leu- |
| | Cys-Lys-Lys-(D)Tyr-Arg-Ser |
| 27 | Cys-Tyr-Phe-Asp-Asp-Ser-Ser-Asn-Val-Leu- |
| | Cys-Lys-Lys-Ala-Arg-Ser |
| 28 | Cys-Tyr-Phe-Asp-Asp-Ser-Ser-Asn-Val-Lys- |
| | Cys-Lys-Lys-(D)Ala-Arg-Ser |
| 29 | Cys-Tyr-Phe-Asp-Asp-Ser-Ser-Asn-Val-Leu- |
| | Cys-Lys-Lys-His-Arg-Ser |
| 30 | Cys-Tyr-Phe-Asp-Asp-Ser-Ser-Asn-Val-Leu- |
| | Cys-Lys-Lys-(D)His-Arg-Ser |
| 31 | [N-terminal: Phenyl-CH₂-CH₂-CO-]Cys-Tyr- |
| | Phe-Asp-Asp-Ser-Ser-Asn-Val-Leu-Cys-Lys- |
| | Lys-Tyr-Arg-Ser |
| 32 | [N-terminal: Phenyl-CH₂-CH₂-CO-]Cys-Tyr- |
| | Phe-Asp-Asp-Ser-Ser-Asn-Val-Leu-Cys-Lys- |
| | Lys-(D)Tyr-Arg-Ser |
| 33 | [N-terminal: Pirazinyl-CO-]Cys-Tyr-Phe- |
| | Asp-Asp-Ser-Ser-Asn-Val-Leu-Cys-Lys- |
| | Lys-Tyr-Arg-Ser |
| 34 | [N-terminal: Pirazinyl-CO-]Cys-Tyr-Phe- |
| | Asp-Asp-Ser-Ser-Asn-Val-Leu-Cys-Lys- |
| | Lys-(D)Tyr-Arg-Ser |
| 35 | Cys-Tyr-Phe-Asn-Asp-Ser-Ser-Gln-Val-Leu- |
| | Cys-Lys-Arg-Tyr-Arg-Ser |
| 36 | Cys-Tyr-Phe-Asn-Asp-Ser-Ser-Gln-Val-Leu- |
| | Cys-Lys-Arg-(D)Tyr-Arg-Ser |
| 37 | Cys-Tyr-Phe-Asp-Asp-Asn-Ser-Asn-Val-Leu- |
| | Cys-Arg-Lys-Tyr-Arg-Ser |
| 38 | Cys-Tyr-Phe-Asp-Asp-Asn-Ser-Asn-Val-Leu- |
| | Cys-Arg-Lys-Tyr-(Me)(D)Arg-Ser |
| 39 | Cys-Tyr-Phe-Asp-Asp-Ser-Ser-Asn-Thr-Leu- |
| | Cys-Lys-Lys-Tyr-Arg-Ser |
| 40 | Cys-Tyr-Phe-Asp-Asp-Ser-Ser-Asn-Thr-Leu- |
| | Cys-Lys-Lys-Tyr-(Me)(D)Arg-Ser |
| 41 | Cys-Tyr-Phe-Asp-Asp-Ser-Ser-Asn-Ala-Leu- |
| | Cys-Lys-Lys-Tyr-Arg-Ser |
| 42 | Cys-Tyr-Phe-Asp-Asp-Ser-Ser-Asn-Ala-Leu- |
| | Cys-Lys-Lys-Tyr-(Me)(D)Arg-Ser |
| 43 | Cys-Tyr-Tyr-Phe-Ser-Ser-Ser-Phe-Val-Leu- |
| | Cys-Lys-Arg-Tyr-Arg-Ser |
| 44 | Cys-Tyr-Tyr-Phe-Ser-Ser-Ser-Phe-Val-Leu- |
| | Cys-Lys-Arg-Tyr-(Me)(D)Arg-Ser |
| 45 | Cys-Tyr-Phe-Phe-Asp-Ser-Ser-Asn-Val-Leu- |
| | Cys-Lys-Lys-Tyr-Arg-Ser |
| 46 | Cys-Tyr-Phe-Phe-Asp-Ser-Ser-Asn-Val-Leu- |
| | Cys-Lys-Lys-(D)Tyr-Arg-Ser |
| 47 | Cys-Tyr-Phe-Nal-Asp-Ser-Ser-Asn-Val-Leu- |
| | Cys-Lys-Lys-Tyr-Arg-Ser |
| 48 | Cys-Tyr-Phe-Nal-Asp-Ser-Ser-Asn-Val-Leu- |
| | Cys-Lys-Lys-(D)Tyr-Arg-Ser |
| 49 | Cys-Tyr-Phe-Asp-Asp-Phe-Ser-Asn-Val-Leu- |
| | Cys-Lys-Lys-Tyr-Arg-Ser |
| 50 | Cys-Tyr-Phe-Asp-Asp-Phe-Ser-Asn-Val-Leu- |
| | Cys-Lys-Lys-(D)Tyr-Arg-Ser |
| 51 | Cys-Tyr-Phe-Asp-Asp-Ser-Phe-Asn-Val-Leu- |
| | Cys-Lys-Lys-Tyr-Arg-Ser |
| 52 | Cys-Tyr-Phe-Asp-Asp-Ser-Phe-Asn-Val-Leu- |
| | Cys-Lys-Lys-(D)Tyr-Arg-Ser |
| 53 | Cys-Tyr-Phe-Asp-Asp-Ser-Ser-Phe-Val-Leu- |
| | Cys-Lys-Lys-Tyr-Arg-Ser |
| 54 | Cys-Tyr-Phe-Asp-Asp-Ser-Ser-Phe-Val-Leu- |
| | Cys-Lys-Lys-(D)Tyr-Arg-Ser |
| 55 | Cys-Tyr-Tyr-Phe-Asn-Ser-Ser-Ser-Val-Leu- |
| | Cys-Lys-Arg-Tyr-Arg-Ser |
| 56 | Cys-Tyr-Tyr-Phe-Asn-Ser-Ser-Ser-Val-Leu- |
| | Cys-Lys-Arg-(D)Tyr-Arg-Ser |
| 57 | Cys-Tyr-Phe-Phe-Asn-Ser-Ser-Gln-Val-Leu- |
| | Cys-Lys-Arg-Tyr-Arg-Ser |
| 58 | Cys-Tyr-Phe-Phe-Asn-Ser-Ser-Gln-Val-Leu- |
| | Cys-Lys-Arg-(D)Tyr-Arg-Ser |
| 59 | Cys-Tyr-Phe-Tyr-Asn-Ser-Ser-Gln-Val-Leu- |
| | Cys-Lys-Arg-Tyr-Arg-Ser |
| 60 | Cys-Tyr-Phe-Tyr-Asn-Ser-Ser-Gln-Val-Leu- |
| | Cys-Lys-Arg-(D)Tyr-Arg-Ser |
| 61 | Cys-Tyr-Tyr-Phe-Asn-Ser-Ser-Gln-Asp-Leu- |
| | Cys-Lys-Arg-Tyr-Arg-Ser |
| 62 | Cys-Tyr-Tyr-Phe-Asn-Ser-Ser-Gln-Asp-Leu- |
| | Cys-Lys-Arg-(D)Tyr-Arg-Ser |
| 63 | Cys-Tyr-Tyr-Phe-Asn-Ser-Ser-Gln-Ser-Leu- |
| | Cys-Lys-Arg-Tyr-Arg-Ser |
| 64 | Cys-Tyr-Tyr-Phe-Asn-Ser-Ser-Gln-Ser-Leu- |
| | Cys-Lys-Arg-(D)Tyr-Arg-Ser |
| 65 | Cys-Tyr-Phe-Asp-Phe-Ser-Ser-Asn-Val-Leu- |
| | Cys-Lys-Lys-Tyr-Arg-Ser |
| 66 | Cys-Tyr-Phe-Asp-Phe-Ser-Ser-Asn-Val-Leu- |
| | Cys-Lys-Lys-(D)TyrArg-Ser |
| 67 | Cys-Tyr-Tyr-Phe-Asn-Ser-Ser-Gln-Val-Leu- |
| | Cys-Lys-Arg-Tyr-Arg-Ser |
| 68 | X₁-Tyr-Tyr-Phe-Ser-Ser-Ser-Phe-Val-Leu- |
| | Cys-Lys-Arg-(D)Tyr-Arg-Ser |
| 69 | CX₂X₃X₄X₅X₆X₇X₈X₉X₁₀CX₁₂X₁₃HisX₁₅X₁₆ |
| 70 | CX₂X₃X₄X₅X₆X₇X₈X₉X₁₀CX₁₂X₁₃(D)HisX₁₅X₁₆ |
| 71 | CX₂X₃X₄X₅X₆X₇X₈X₉X₁₀CX₁₂X₁₃AlaX₁₅X₁₆ |
| 72 | CX₂X₃X₄X₅X₆X₇X₈X₉X₁₀CX₁₂X₁₃(D)AlaX₁₅X₁₆ |

The following conventions have been used in referencing the sequences herein, including SEQ ID NOs: 1-72:

The standard single letter amino acid codes for the 20 naturally occurring amino acids. Parentheses encompass non-natural amino acid descriptors, e.g., (D)Tyr, which stands for D-tyrosine, (Nα-Me)Arg which stands for Nα-Me-Arg

In all cases the peptides can be cyclized using disulfide bonds. Cys at position 1 is disulfide bonded to the Cys at position 11.

In certain embodiments, the peptide of the invention comprises the general structure shown in SEQ ID NO:19: CX₂X₃X₄X₅X₆X₇X₈X₉X₁₀CX₁₂X₁₃X₁₄X₁₅X₁₆, wherein X₁-X₁₆ vary independently of each other, and wherein X can be any naturally occurring or non-naturally occurring amino acid, and wherein the peptide includes at least two Cys residues at positions 1 and 11, and wherein up to 6 amino acids may be absent. The amino acids that are absent may be contiguous or discontiguous.

In another embodiment, the peptide of the invention comprises the general structure shown in SEQ ID NO:20: CX₂X₃X₄X₅X₆X₇X₈X₉X₁₀CX₁₂X₁₃(D) X₁₄X₁₅X₁₆, wherein X₁-X₁₆ vary independently of each other, and wherein X can be any naturally occurring or non-naturally occurring amino acid, and wherein the peptide includes at least two Cys residues at positions 1 and 11, wherein position 14 includes a stabilizing non-naturally occurring amino acid, and wherein up to 6 amino acids may be absent. The amino acids that are absent may be contiguous or discontiguous. In one embodiment, position 14 is (D)Tyr. In another embodiment, position 15 is (D)Arg or (Nα-Me)Arg.

Accordingly, in another embodiment, the peptide of the invention comprises the general structure shown in SEQ ID NO:21: CX₂X₃X₄X₅X₆X₇X₈X₉X₁₀CX₁₂X₁₃(D)TyrX₁₅X₁₆, wherein X₁-X₁₆ vary independently of each other, and wherein X can be any naturally occurring or non-naturally occurring amino acid, and wherein the peptide includes at least two Cys residues at positions 1 and 11, and a (D)Tyr at position 14.

Accordingly, in another embodiment, the peptide of the invention comprises the general structure shown in SEQ ID NO:22: CX₂X₃X₄X₅X₆X₇X₈X₉X₁₀CX₁₂X₁₃(Nα-Me)ArgX₁₅X₁₆, wherein X₁-X₁₆ vary independently of each other, and wherein X can be any naturally occurring or non-naturally occurring amino acid, and wherein the peptide includes at least two Cys residues at positions 1 and 11, and a (Nα-Me)Arg at position 14.

In another embodiment, the peptide of the invention comprises the general structure shown in SEQ ID NO:24: CX₂X₃X₄X₅X₆X₇X₈X₉X₁₀CX₁₂X₁₃X₁₄X₁₅X₁₆, wherein X₁-X₁₆ vary independently of each other, and wherein X can be any naturally occurring or non-naturally occurring amino acid, and wherein the peptide includes at least two Cys residues at positions 1 and 11, (D)Arg or (Nα-Me)Arg at position 15, and wherein up to 6 amino acids may be absent. The amino acids that are absent may be contiguous or discontiguous.

In another embodiment, the peptide of the invention comprises the general structure shown in SEQ ID NO:24: CX₂X₃X₄X₅X₆X₇X₈X₉X₁₀CX₁₂(D)lysX₁₄X₁₅X₁₆, wherein X₁-X₁₆ vary independently of each other, and wherein X can be any naturally occurring or non-naturally occurring amino acid, and wherein the peptide includes at least two Cys residues at positions 1 and 11, (D)lys at position 13, and wherein up to 6 amino acids may be absent. The amino acids that are absent may be contiguous or discontiguous.

In certain embodiments, the peptide of the invention comprises the amino acid sequence set forth as any one of SEQ ID NOs:1-72. In further embodiments, the peptide has at least 50% identity to any one of SEQ ID NOs: 1-72. In other further embodiments, the peptide has between 50%-99% identity to any one of SEQ ID NOs:1-72. For example, the peptide can have 50%, 51%, 52%, 53%, 54%, 55%, 56%, 57%, 58%, 59%, 0%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to any one of SEQ ID NOs:1-72. In certain embodiments, the peptide of the invention comprises the amino acid sequence set forth as SEQ ID NO:25. In further embodiments, the peptide has at least 50% identity to SEQ ID NO:25. In other further embodiments, the peptide has between 50%-99% identity to SEQ ID NO:25. For example, the peptide can have 50%, 51%, 52%, 53%, 54%, 55%, 56%, 57%, 58%, 59%, 0%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to SEQ ID NO:25.

In other embodiments, the peptides of the invention can include any suitable variants, analogs, homologs, or fragments of those peptides of SEQ ID NOs:1-72 as shown below.

In certain embodiments, the peptide comprises SEQ ID NO:1

Cys-Tyr-Phe-Asp-Asp-Ser-Ser-Asn-Val-Leu-Cys-Lys-
Lys-Tyr-Arg-Ser.

In certain embodiments, the peptide comprises SEQ ID NO:2

Cys-Tyr-Phe-Asp-Asp-Ser-Ser-Asn-Val-Leu-Cys-Lys-
(D)lys-Tyr-Arg-Ser.

In certain embodiments, the peptide comprises SEQ ID NO:3

Cys-Tyr-Phe-Asp-Asp-Ser-Ser-Asn-Val-Leu-Cys-Lys-
Lys-(D)tyr-Arg-Ser.

In certain embodiments, the peptide comprises SEQ ID NO:4

Cys-Tyr-Phe-Asp-Asp-Ser-Ser-Asn-Val-Leu-Cys-Lys-
Lys-Tyr-(D)arg-Ser.

In certain embodiments, the peptide comprises SEQ ID NO:5

Cys-Tyr-Phe-Asp-Asp-Ser-Ser-Asn-Val-Leu-Cys-Lys-
Lys-Tyr-(Nα-Me)Arg-Ser.

In certain embodiments, the peptide comprises SEQ ID NO:6

Cys-Tyr-Phe-Asp-Asp-Ser-Ser-Asn-Val-Leu-Cys.

In certain embodiments, the peptide comprises SEQ ID NO:7

Cys-Tyr-Phe-Asp-Asp-Ser-Ser-Asn-Val-Leu-Cys-Lys-
Lys-Tyr.

In certain embodiments, the peptide comprises SEQ ID NO:8

Cys-Tyr-Phe-Asp-Asp-Ser-Ser-Asn-Val-Leu-Cys-Lys.

In certain embodiments, the peptide comprises SEQ ID NO:9

Cys-Tyr-Phe-Asp-Asp-Ser-Ser-Asn-Val-Leu-Cys-Lys-
Lys.

In certain embodiments, the peptide comprises SEQ ID NO:10

Cys-Tyr-Phe-Asp-Asp-Ser-Ser-Asn-Val-Leu-Cys-Lys-
Arg-Tyr-Arg-Ser.

In certain embodiments, the peptide comprises SEQ ID NO:11

Cys-Tyr-Phe-Asp-Asp-Ser-Ser-Asn-Val-Leu-Cys-Lys-
Arg-D-tyr-Arg-Ser.

In certain embodiments, the peptide comprises SEQ ID NO:12

Cys-Tyr-Tyr-Asp-Asp-Ser-Ser-Ser-Val-Leu-Cys-Lys-
Lys-Tyr-Arg-Ser.

In certain embodiments, the peptide comprises SEQ ID NO:12

Cys-Tyr-Tyr-Asp-Asp-Ser-Ser-Ser-Val-Leu-Cys-Lys-
Lys-(D)Tyr-Arg-Ser.

In certain embodiments, the peptide comprises SEQ ID NO:14

Cys-Tyr-Tyr-Asp-Asn-Ser-Ser-Ser-Val-Leu-Cys-Lys-
Lys-Tyr-Arg-Ser.

In certain embodiments, the peptide comprises SEQ ID NO:15

Cys-Tyr-Tyr-Asp-Asn-Ser-Ser-Ser-Val-Leu-Cys-Lys-
Lys-(D)Tyr-Arg-Ser.

In certain embodiments, the peptide comprises SEQ ID NO:16

Cys-Tyr-Tyr-Asp-Asn-Ser-Ser-Ser-Val-Leu-Cys-Lys-
Arg-Tyr-Arg-Ser.

In certain embodiments, the peptide comprises SEQ ID NO:17

Cys-Tyr-Tyr-Asp-Asn-Ser-Ser-Ser-Val-Leu-Cys-Lys-
Arg-(D)Tyr-Arg-Ser.

In certain embodiments, the peptide comprises SEQ ID NO:18

Cys-Tyr-Tyr-Asp-Asn-Ser-Ser-Ser-Val-Leu-Cys-Lys-
Arg-Tyr-(Nα-Me)Arg-Ser.

In certain embodiments, the peptide comprises SEQ ID NO:25:

Cys-Tyr-Tyr-Phe-Asn-Ser-Ser-Gln-Val-Leu-Cys-
Lys-Arg-(D)Tyr-Arg-Ser.

In certain embodiments, the peptide comprises SEQ ID NO:19:

CX₂X₃X₄X₅X₆X₇X₈X₉X₁₀CX₁₂X₁₃X₁₄X₁₅X_{16.}

In certain embodiments, the peptide comprises SEQ ID NO:20:

CX₂X₃X₄X₅X₆X₇X₈X₉X₁₀CX₁₂X₁₃(D)X₁₄X₁₅X_{16.}

In certain embodiments, the peptide comprises SEQ ID NO:21:

CX₂X₃X₄X₅X₆X₇X₈X₉X₁₀CX₁₂X₁₃(D)TyrX₁₅X_{16.}

In certain embodiments, the peptide comprises SEQ ID NO:22:

CX₂X₃X₄X₅X₆X₇X₈X₉X₁₀CX₁₂X₁₃(Nα-Me)ArgX₁₅X_{16.}

In certain embodiments, the peptide comprises SEQ ID NO:23:

CX₂X₃X₄X₅X₆X₇X₈X₉X₁₀CX₁₂X₁₃X₁₄X₁₅X_{16.}

In certain embodiments, the peptide comprises SEQ ID NO:24:

CX₂X₃X₄X₅X₆X₇X₈X₉X₁₀CX₁₂(D)lysX₁₄X₁₅X_{16.}

In certain embodiments, the peptide comprises SEQ ID NO:26:

Cys-Tyr-Phe-Asp-Asp-Ser-Ser-Asn-Val-Leu-Cys-Lys-
Lys-(D)Tyr-Arg-Ser

In certain embodiments, the peptide comprises SEQ ID NO:27:

Cys-Tyr-Phe-Asp-Asp-Ser-Ser-Asn-Val-Leu-Cys-Lys-
Lys-Ala-Arg-Ser

In certain embodiments, the peptide comprises SEQ ID NO:28:

Cys-Tyr-Phe-Asp-Asp-Ser-Ser-Asn-Val-Lys-Cys-Lys-
Lys-(D)Ala-Arg-Ser

In certain embodiments, the peptide comprises SEQ ID NO:29:

Cys-Tyr-Phe-Asp-Asp-Ser-Ser-Asn-Val-Leu-Cys-Lys-
Lys-His-Arg-Ser

In certain embodiments, the peptide comprises SEQ ID NO:30:

Cys-Tyr-Phe-Asp-Asp-Ser-Ser-Asn-Val-Leu-Cys-Lys-
Lys-(D)His-Arg-Ser

In certain embodiments, the peptide comprises SEQ ID NO:31:

[N-terminal: Phenyl-CH₂-CH₂-CO-]Cys-Tyr-Phe-Asp-
Asp-Ser-Ser-Asn-Val-Leu-Cys-Lys-Lys-Tyr-Arg-Ser

In certain embodiments, the peptide comprises SEQ ID NO:32:

[N-terminal: Phenyl-CH₂-CH₂-CO-]Cys-Tyr-Phe-Asp-
Asp-Ser-Ser-Asn-Val-Leu-Cys-Lys-Lys-(D)Tyr-Arg-Ser

In certain embodiments, the peptide comprises SEQ ID NO:33:

[N-terminal: Pirazinyl-CO-]Cys-Tyr-Phe-Asp-Asp-
Ser-Ser-Asn-Val-Leu-Cys-Lys-Lys-Tyr-Arg-Ser

In certain embodiments, the peptide comprises SEQ ID NO:34:

[N-terminal: Pirazinyl-CO-]Cys-Tyr-Phe-Asp-Asp-
Ser-Ser-Asn-Val-Leu-Cys-Lys-Lys-(D)Tyr-Arg-Ser

In certain embodiments, the peptide comprises SEQ ID NO:35:

Cys-Tyr-Phe-Asn-Asp-Ser-Ser-Gln-Val-Leu-Cys-Lys-
Arg-Tyr-Arg-Ser

In certain embodiments, the peptide comprises SEQ ID NO:36:

Cys-Tyr-Phe-Asn-Asp-Ser-Ser-Gln-Val-Leu-Cys-Lys-
Arg-(D)Tyr-Arg-Ser

In certain embodiments, the peptide comprises SEQ ID NO:37:

Cys-Tyr-Phe-Asp-Asp-Asn-Ser-Asn-Val-Leu-Cys-Arg-
Lys-Tyr-Arg-Ser

In certain embodiments, the peptide comprises SEQ ID NO:38:

Cys-Tyr-Phe-Asp-Asp-Asn-Ser-Asn-Val-Leu-Cys-Arg-
Lys-Tyr-(Me)(D)Arg-Ser

In certain embodiments, the peptide comprises SEQ ID NO:39:

Cys-Tyr-Phe-Asp-Asp-Ser-Ser-Asn-Thr-Leu-Cys-Lys-
Lys-Tyr-Arg-Ser

In certain embodiments, the peptide comprises SEQ ID NO:40:

Cys-Tyr-Phe-Asp-Asp-Ser-Ser-Asn-Thr-Leu-Cys-Lys-
Lys-Tyr-(Me)(D)Arg-Ser

In certain embodiments, the peptide comprises SEQ ID NO:41:

Cys-Tyr-Phe-Asp-Asp-Ser-Ser-Asn-Ala-Leu-Cys-Lys-
Lys-Tyr-Arg-Ser

In certain embodiments, the peptide comprises SEQ ID NO:42:

Cys-Tyr-Phe-Asp-Asp-Ser-Ser-Asn-Ala-Leu-Cys-Lys-
Lys-Tyr-(Me)(D)Arg-Ser

In certain embodiments, the peptide comprises SEQ ID NO:43:

Cys-Tyr-Tyr-Phe-Ser-Ser-Ser-Phe-Val-Leu-Cys-Lys-
Arg-Tyr-Arg-Ser

In certain embodiments, the peptide comprises SEQ ID NO:44:

Cys-Tyr-Tyr-Phe-Ser-Ser-Ser-Phe-Val-Leu-Cys-Lys-
Arg-Tyr-(Me)(D)Arg-Ser

In certain embodiments, the peptide comprises SEQ ID NO:45:

Cys-Tyr-Phe-Phe-Asp-Ser-Ser-Asn-Val-Leu-Cys-Lys-
Lys-Tyr-Arg-Ser

In certain embodiments, the peptide comprises SEQ ID NO:46:

Cys-Tyr-Phe-Phe-Asp-Ser-Ser-Asn-Val-Leu_Cys-Lys-
Lys-(D)Tyr-Arg-Ser

In certain embodiments, the peptide comprises SEQ ID NO:47:

Cys-Tyr-Phe-Nal-Asp-Ser-Ser-Asn-Val-Leu-Cys-Lys-
Lys-Tyr-Arg-Ser

In certain embodiments, the peptide comprises SEQ ID NO:48:

Cys-Tyr-Phe-Nal-Asp-Ser-Ser-Asn-Val-Leu-Cys-Lys-
Lys-(D)Tyr-Arg-Ser

In certain embodiments, the peptide comprises SEQ ID NO:49:

Cys-Tyr-Phe-Asp-Asp-Phe-Ser-Asn-Val-Leu-Cys-Lys-
Lys-Tyr-Arg-Ser

In certain embodiments, the peptide comprises SEQ ID NO:50:

Cys-Tyr-Phe-Asp-Asp-Phe-Ser-Asn-Val-Leu-Cys-Lys-
Lys-(D)Tyr-Arg-Ser

In certain embodiments, the peptide comprises SEQ ID NO:51:

Cys-Tyr-Phe-Asp-Asp-Ser-Phe-Asn-Val-Leu-Cys-Lys-
Lys-Tyr-Arg-Ser

In certain embodiments, the peptide comprises SEQ ID NO:52:

Cys-Tyr-Phe-Asp-Asp-Ser-Phe-Asn-Val-Leu-Cys-Lys-
Lys-(D)Tyr-Arg-Ser

In certain embodiments, the peptide comprises SEQ ID NO:53:

Cys-Tyr-Phe-Asp-Asp-Ser-Ser-Phe-Val-Leu-Cys-Lys-
Lys-Tyr-Arg-Ser

In certain embodiments, the peptide comprises SEQ ID NO:54:

Cys-Tyr-Phe-Asp-Asp-Ser-Ser-Phe-Val-Leu-Cys-Lys-
Lys-(D)Tyr-Arg-Ser

In certain embodiments, the peptide comprises SEQ ID NO:55:

Cys-Tyr-Tyr-Phe-Asn-Ser-Ser-Ser-Val-Leu-Cys-Lys-
Arg-Tyr-Arg-Ser

In certain embodiments, the peptide comprises SEQ ID NO:56:

Cys-Tyr-Tyr-Phe-Asn-Ser-Ser-Ser-Val-Leu-Cys-Lys-
Arg-(D)Tyr-Arg-Ser

In certain embodiments, the peptide comprises SEQ ID NO:57:

Cys-Tyr-Phe-Phe-Asn-Ser-Ser-Gln-Val-Leu-Cys-Lys-
Arg-Tyr-Arg-Ser

In certain embodiments, the peptide comprises SEQ ID NO:58:

Cys-Tyr-Phe-Phe-Asn-Ser-Ser-Gln-Val-Leu-Cys-Lys-
Arg-(D)Tyr-Arg-Ser

In certain embodiments, the peptide comprises SEQ ID NO:59:

Cys-Tyr-Phe-Tyr-Asn-Ser-Ser-Gln-Val-Leu-Cys-Lys-
Arg-Tyr-Arg-Ser

In certain embodiments, the peptide comprises SEQ ID NO:60:

Cys-Tyr-Phe-Tyr-Asn-Ser-Ser-Gln-Val-Leu-Cys-Lys-
Arg-(D)Tyr-Arg-Ser

In certain embodiments, the peptide comprises SEQ ID NO:61:

Cys-Tyr-Tyr-Phe-Asn-Ser-Ser-Gln-Asp-Leu-Cys-Lys-
Arg-Tyr-Arg-Ser

In certain embodiments, the peptide comprises SEQ ID NO:62:

Cys-Tyr-Tyr-Phe-Asn-Ser-Ser-Gln-Asp-Leu-Cys-Lys-
Arg-(D)Tyr-Arg-Ser

In certain embodiments, the peptide comprises SEQ ID NO:63:

Cys-Tyr-Tyr-Phe-Asn-Ser-Ser-Gln-Ser-Leu-Cys-Lys-
Arg-Tyr-Arg-Ser

In certain embodiments, the peptide comprises SEQ ID NO:64:

Cys-Tyr-Tyr-Phe-Asn-Ser-Ser-Gln-Ser-Leu-Cys-Lys-
Arg-(D)Tyr-Arg-Ser

In certain embodiments, the peptide comprises SEQ ID NO:65:

Cys-Tyr-Phe-Asp-Phe-Ser-Ser-Asn-Val-Leu-Cys-Lys-
Lys-Tyr-Arg-Ser

In certain embodiments, the peptide comprises SEQ ID NO:66:

Cys-Tyr-Phe-Asp-Phe-Ser-Ser-Asn-Val-Leu-Cys-Lys-
Lys-(D)TyrArg-Ser

In certain embodiments, the peptide comprises SEQ ID NO:67:

Cys-Tyr-Tyr-Phe-Asn-Ser-Ser-Gln-Val-Leu-Cys-Lys-
Arg-Tyr-Arg-Ser

In certain embodiments, the peptide comprises SEQ ID NO:68:

X₁-Tyr-Tyr-Phe-Ser-Ser-Ser-Phe-Val-Leu-Cys-Lys-
Arg-(D)Tyr-Arg-Ser,

wherein X₁ can be no amino acid or any naturally occurring or non-naturally occurring amino acid.

In certain embodiments, the peptide comprises SEQ ID NO:69:

CX₂X₃X₄X₅X₆X₇X₈X₉X₁₀CX₁₂X₁₃HisX₁₅X₁₆,

wherein X₁-X₁₆ vary independently of each other, and wherein X can be any naturally occurring or non-naturally occurring amino acid.

In certain embodiments, the peptide comprises SEQ ID NO:70:

CX₂X₃X₄X₅X₆X₇X₈X₉X₁₀CX₁₂X₁₃(D)HisX₁₅X₁₆,

wherein X₁-X₁₆ vary independently of each other, and wherein X can be any naturally occurring or non-naturally occurring amino acid.

In certain embodiments, the peptide comprises SEQ ID NO:71:

CX₂X₃X₄X₅X₆X₇X₈X₉X₁₀CX₁₂X₁₃AlaX₁₅X₁₆,

wherein X₁-X₁₆ vary independently of each other, and wherein X can be any naturally occurring or non-naturally occurring amino acid.

In certain embodiments, the peptide comprises SEQ ID NO:72:

CX₂X₃X₄X₅X₆X₇X₈X₉X₁₀CX₁₂X₁₃(D)AlaX₁₅X₁₆,

wherein X₁-X₁₆ vary independently of each other, and wherein X can be any naturally occurring or non-naturally occurring amino acid.

In the case of peptide sequences which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g., in sequence, in structure, or in function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

The invention also provides for compounds having altered sequences including insertions such that the overall amino acid sequence is lengthened, while the compound still retains the appropriate properties. Additionally, altered sequences may include random or designed internal deletions that truncate the overall amino acid sequence of the compound, however the compound still retains its functional properties. In certain embodiments, one or more amino acid residues within SEQ ID NOs:1-72 are replaced with other amino acid residues having physical and/or chemical properties similar to the residues they are replacing. Preferably, conservative amino acid substitutions are those wherein an amino acid is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the properties of the altered compound.

It is also contemplated that the peptides of the invention may be provided in the form of a propeptide or propolypeptide. For purposes of the invention, a propeptide or propolypeptide refers to a precursor version or variant of a peptide of the invention that is substantially inactive as compared to the mature form of the peptide, that further includes a cleavable or otherwise removable portion. The precursor form of the peptides of the invention preferably do not have activity or that the activity of the peptide is subdued or otherwise reduced. Such precursor forms can include cleavable moieties or extended amino acid sequences, e.g., a leader sequence or a terminal polypeptide sequence, that may be useful for a variety of reasons, for example, in cell secretion during cellular production of a peptide of the invention, or for masking the activity of a peptide of the invention until the propeptide or propolypeptide encounters the target injury site of action. For example, the propeptide or propolypeptide may contain a cleavable moiety to remove a masking portion or leader portion which is removable only within the diseased tissue due to a heightened activity (e.g. protease or enzyme) that is characteristic only of the diseased state and not present in a healthy tissue. Such masks and leader sequences are known in the art. In this way, the peptides of the invention can be “targeted” with increased specificity for the desired site of treatment.

The peptides of the present invention may be pegylated, or modified, e.g., branching, at any amino acid residue containing a reactive side chain, e.g., lysine residue, or chemically reactive group on the linker. The peptides of the present invention may be linear or cyclized. The tail sequence of the peptides may vary in length.

In certain embodiments, the compounds of the invention can include commonly encountered amino acids which are not genetically encoded. These non-genetically encoded amino acids include, but are not limited to, beta-alanine (beta-Ala) and other omega-amino acids such as 3-aminopropionic acid (Dap), 2,3-diaminopropionic acid (Dpr), 4-aminobutyric acid and so forth; alpha-aminoisobutyric acid (Aib); epsilon-aminohexanoic acid (Aha); delta-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); beta-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH2)); N-methyl valine (MeVal); homocysteine (hCys) and homoserine (hSer). Non-naturally occurring variants of the compounds may be produced by mutagenesis techniques or by direct synthesis.

Methods of Making the Peptides of the Invention

In another aspect, the present invention relates to methods for making the peptides of the invention. Such methods, in general, can include any suitable known method in the art for conducting such tasks, including synthetic peptide chemistry, recombinant expression of the peptides of the invention using appropriate prokaryotic or eukaryotic host cells and expression systems, or recombinant expression of the peptides as a feature of somatic gene transfer, i.e., expression as part of the administration regimen at the site of treatment.

In one embodiment, a peptide can be synthesized chemically using standard peptide synthesis techniques, e.g., solid-phase or solution-phase peptide synthesis. That is, the compounds disclosed as SEQ ID NOs:1-72 may be chemically synthesized, for example, on a solid support or in solution using compositions and methods well known in the art, see, e.g., Fields, G. B. (1997) Solid-Phase Peptide Synthesis. Academic Press, San Diego, incorporated by reference in its entirety herein.

The biological activity, of the peptides of the invention can be characterized using any conventional in vivo and in vitro assays that have been developed to measure the biological activity of this class of peptides.

Pharmaceutical Compositions

The peptides and/or nucleic acid molecules of the invention, and derivatives, fragments, analogs and homologs thereof, can be incorporated into pharmaceutical compositions suitable for administration. Such compositions typically comprise the nucleic acid molecule, polypeptide, or antibody with or without a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fingi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition a compound, which delays absorption, for example, aluminum monostearate and gelatin.

Sterile injectable solutions can be prepared by incorporating the peptide in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fasidic acid derivatives. Transmucosal administration can be accomplished through the use of nasal sprays or suppositories. For transdermal administration, the active compounds are formulated into ointments, salves, gels, or creams as generally known in the art.

The compounds can also be prepared as pharmaceutical compositions in the form of suppositories (e.g., with conventional suppository bases such as cocoa butter and other glycerides) or retention enemas for rectal delivery. The compounds can be prepared for use in conditioning or treatment of ex vivo explants or implants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated by reference in its entirety herein.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The present invention also contemplates pharmaceutical compositions and formulations for co-administering the peptides of the invention with one or more additional active agents. The one or more additional active agents can include other agents or therapies for treating a kidney disease or disorder. The one or more additional active agents can also include other therapies relating to the underlying disease or condition that results in or is involved in or relates to the kidney disease or disorder.

Methods of Treatment

The invention provides for both prophylactic and therapeutic methods of treating a subject having a disease or disorder associated with aberrant TDF polypeptide or TDFRP compound target molecule expression or activity, or a method of delaying the progression of a disorder associated with aberrant TDF polypeptide or TDFRP compound target molecule expression or activity. TDF and TDFRP compound target molecules, such as TDF receptors, play a role in cell differentiation. Cell differentiation is the central characteristic of tissue morphogenesis. Tissue morphogenesis is a process involved in adult tissue repair and regeneration mechanisms. The degree of morphogenesis in adult tissue varies among different tissues and is related, among other things, to the degree of cell turnover in a given tissue.

The bone morphogenetic proteins are members of the transforming growth factor-beta superfamily. Ozkaynak et al. (EMBO J. 9: 2085-2093, 1990) purified a novel bovine osteogenic protein homolog, which they termed ‘osteogenic protein-1’ (OP-1; a.k.a., BMP-7). The authors used peptide sequences to clone the human genomic and cDNA clones of OP-1, later named BMP-7. The BMP-7 cDNAs predicted a 431-amino acid polypeptide that includes a secretory signal sequence. The TDFRP compounds described herein are structural mimetics of the biologically active regions of bone morphogenic proteins, for example, but not limited to, BMP-7 (OP-1), and related peptides. Biologically active regions include, for example, the Finger 1 and Finger 2 regions of BMP-7. Groppe et al. (Nature 420: 636-642, 2002) reported the crystal structure of the antagonist Noggin (602991) bound to BMP-7.

TDFRP compounds are useful to treat diseases and disorders that are amenable to treatment with BMP polypeptides. The TDFRP compounds of the invention are useful to alter, e.g., inhibit or accelerate, the ability to repair and regenerate diseased or damaged tissues and organs, as well as, to treat TDF-associated disorders. Particularly useful areas for TDFRP-based human and veterinary therapeutics include reconstructive surgery, the treatment of tissue degenerative diseases including, for example, renal disease, brain trauma, stroke, atherosclerosis, arthritis, emphysema, osteoporosis, cardiomyopathy, cirrhosis, degenerative nerve diseases, inflammatory diseases, and cancer, and in the regeneration of tissues, organs and limbs. The TDFRP compounds of the invention can also be used to promote or inhibit the growth and differentiation of muscle, bone, skin, epithelial, heart, nerve, endocrine, vessel, cartilage, periodontal, liver, retinal, and connective tissue, or any tissue where functional TDRFP compound target molecules are expressed. Accordingly, diseases associated with aberrant TDF polypeptide or TDFRP compound target molecule expression include viral infections, cancer, healing, neurodegenerative disorders, e.g., Alzheimer's Disease, Parkinson's Disorder, immune disorders, and bone disorders. For example, TDFRP-based therapeutic compositions are useful to induce regenerative healing of bone defects such as fractures, as well as, to preserve or restoring healthy metabolic properties in diseased tissue, e.g., osteopenic bone tissue.

Marker et al. (Genomics 28: 576-580, 1995) studied the distribution of BMP-7 transcripts at various anatomical sites disrupted by Holt-Oram syndrome (142900) mutations. They found BMP-7 expression in all structures that are altered in Holt-Oram patients, including the heart, proximal and distal forelimb, clavicle, and scapula, as well as other unaffected tissues. Solursh et al. (Biochem. Biophys. Res. Commun. 218: 438-443, 1996) examined developmental and temporal expression of OP-1 by hybridization with histologic sections of rat embryos during a 3-day period comprising the primitive streak stages to early limb bud stages. OP-1 expression was detected in the neuroepithelium of the optic vesicle at day E11.5 and was limited to the presumptive neural retina and developing lens placode. From E12.5-E13.5, they found expression in the neural retina, lens, and developing cornea.

You and Kruse (Invest. Ophthal. Vis. Sci. 43: 72-81, 2002) studied corneal myofibroblast differentiation and signal transduction induced by the TGF-β family members activin A and BMP-7. They found that activin A induced phosphorylation of SMAD2, and BMP-7 induced SMAD1, both of which were inhibited by follistatin. The TGF-β proteins have different functions in the cornea.

TDFRP compounds can be used in the prophylaxis or treatment of coronary atherosclerosis. Induction of BMPs and subsequent inhibition of vascular smooth muscle cell growth and/or induction of vascular bone formation can contribute to the mechanisms by which statins increase plaque stability in patients with coronary atherosclerosis (Emmanuele et al., Biochem Biophys Res Commun. 2003 Feb. 28; 302(1):67-72). Further, studies by Davies et al., (J Am Soc Nephrol. 2003 June; 14(6):1559-67) are consistent with BMP-7 deficiency as a pathophysiologic factor in chronic renal failure, and demonstrate its efficacy as a potential treatment of vascular calcification.

TDFRP compounds can be used to treat cancer, e.g., breast cancer and prostate cancer. Schwalbe et al., (Int J Oncol. 2003 July; 23(1):89-95) analyzed normal breast tissue and tumor tissue samples from 170 invasive ductal carcinomas of the breast by immunohistochemistry. BMP-7 expression was observed in normal breast tissue in the end buds, but not in the ductus lactiferus. BMP-7 protein was detected in all 170 tumor samples. The expression of BMP-7 was highly correlated with estrogen receptor levels (p<l=0.01) and progesterone receptor levels (p<l=0.01), which are important markers for breast cancer prognosis and therapy. Further, Masuda et al., (Prostate. 2003 Mar. 1; 54(4):268-74) demonstrated increased expression of bone morphogenetic protein-7 in bone metastatic prostate cancer.

TDFRP compounds can be used to treat renal dysfunction, disease and injury, e.g. ureteral obstruction, acute and chronic renal failure, renal fibrosis, and diabetic nephropathy. (Klar, S., J. Nephrol. 2003 March-April; 16(2):179-85) demonstrated that BMP-7 treatment significantly decreased renal injury in a rat model of ureteral obstruction (UUO), when treatment was initiated at the time of injury. Subsequent studies suggested that BMP-7 treatment also attenuated renal fibrosis when administered after renal fibrosis had begun. This treatment protocol was also found to increase significantly renal function from the levels measured in the vehicle-treated group. BMP-7 also partially reversed the diabetic nephropathy induced in rats by a single dose of Streptozotocin. It restored glomerular filtration rate (GFR), decreased the excretion of protein, and restored histology towards normal. TDFRP can be used in the prophylaxis or treatment of renal disease, e.g., chronic renal failure. Studies by Klahr et al., (Kidney Int Suppl. 2002 May; (80):23-6) indicate that administration of BMP-7 maintains and restores renal function and structure in animals with ureteral obstruction and diabetic nephropathy.

TDFRP compounds can be used in the prophylaxis or treatment of diabetic nephropathy. Wang et al., (Kidney Int. 2003 June; 63(6):2037-49) have shown that BMP-7 partially reversed diabetic-induced kidney hypertrophy, restoring GFR, urine albumin excretion, and glomerular histology toward normal. Restoration of BMP-7 expression was associated with a successful repair reaction and a reversal of the ill-fated injury response.

TDFRP compounds can be used in the prophylaxis or treatment of renal fibrosis. Exogenous administration of recombinant human bone morphogenetic protein (BMP)-7 was recently shown to ameliorate renal glomerular and interstitial fibrosis in rodents with experimental renal diseases (Wang and Hirschberg, Am J Physiol Renal Physiol. 2003 May; 284(5):F1006-13).

Alport syndrome is a genetic disorder resulting from mutations in type IV collagen genes. The defect results in pathological changes in kidney glomerular and inner-ear basement membranes. In the kidney, progressive glomerulonephritis culminates in tubulointerstitial fibrosis and death. Using gene knockout-mouse models, it has been shown that two different pathways, one mediated by transforming growth factor (TGF)-β1 and the other by integrin alpha1beta1, affect Alport glomerular pathogenesis in distinct ways. The mRNAs encoding TGF-β1 (in both mouse and human), entactin, fibronectin, and the collagen alpha1(IV) and alpha2(IV) chains are significantly induced in total kidney as a function of Alport renal disease progression. The induction of these specific mRNAs is observed in the glomerular podocytes of animals with advanced disease. Type IV collagen, laminin-1, and fibronectin are shown to be elevated in the tubulointerstitium at 10 weeks, but not at 6 weeks, suggesting that elevated expression of specific mRNAs on Northern blots reflects events associated with tubulointerstitial fibrosis. Thus, concomitant accumulation of mRNAs encoding TGF-β1 and extracellular matrix components in the podocytes of diseased kidneys may reflect key events in Alport renal disease progression. Importantly, TGF-β1 appears to have a critical role in both glomerular and tubulointerstitial damage associated with Alport syndrome. Recently, BMP-7 has been implicated to reverse the renal pathology caused by TGF-β in Alport renal disease progress. BMP-7 inhibits tubular epithelial cell de-differentiation, mesenchymal transformation and apoptosis stimulated by various renal injuries. Also it preserves glomerular integrity and inhibits injury-mediated mesangial matrix accumulation. BMP-7 may be a powerful new therapeutic agent for chronic kidney disease, with the novel attribute of not only treating the kidney disease itself, but also directly inhibiting some of the most important complications of the Alport syndrome.

TDFRP compounds can be used to facilitate tissue repair. Grande et al., (J Bone Joint Surg Am. 2003; 85-A Suppl 2:111-6) demonstrated that the addition of either the BMP-7 or the Shh gene significantly enhanced the quality of the repair tissue, resulting in a much smoother surface and more hyaline-appearing cartilage. There was, however, a noticeable difference in the persistence of the cartilage phase between the group that received the Shh gene and the group that received the BMP-7 gene, with the subchondral compartment in the latter group seeming to remodel with bone much faster

TDFRP compounds can be used to in the prophylaxis or treatment of diseases of the oral cavity, e.g., by affecting direct capping of bioactive molecules, or inducing the formation of reparative dentin and coronal or radicular pulp mineralization (Goldberg et al., Am J Dent. 2003 February; 16(1):66-76). Further, TDFRP can be used in the prophylaxis or treatment of periodontal disease. Osseous lesions treated by Ad-BMP-7 gene delivery demonstrated rapid chrondrogenesis, with subsequent osteogenesis, cementogenesis and predictable bridging of the periodontal bone defects. These results demonstrate successful evidence of periodontal tissue engineering using ex vivo gene transfer of BMPs and offers a new approach for repairing periodontal defects (Jin et al., J Periodontol. 2003 February; 74(2):202-13).

TDFRP compounds can be used in the prophylaxis or treatment of traumatic brain injury, e.g., stroke, see, e.g., Cairns and Finkelstein, Phys Med Rehabil Clin N Am. 2003 February; 14(1 Suppl):S 135-42). Intravenous administration of BMP-7 after ischemia improves motor function in stroke rats (Chang et al., Stroke. 2003 February; 34(2):558-64) and the TDFRP compounds may protect against or repair reperfusion injury. Further, Chang et al., (Neuropharmacology 2002 September; 43(3):418-26) have demonstrated that bone morphogenetic proteins are involved in fetal kidney tissue transplantation-induced neuroprotection in stroke rats. Rehabilitation after cell-based cartilage repair can be prolonged, leading to decreased patient productivity and quality of life by treating a subject with TDFRP compounds. Implantation of genetically modified chondrocytes expressing BMP-7 accelerates the appearance of hyaline-like repair tissue in experimental cartilage defects and that might be important cartilage homeostasis (Hidaka et al., J Orthop Res. 2003 July; 21(4):573-83). The synovium is a thin tissue lining the nonarticular surfaces of diarthrodial joints. Synovial tissues contain various types of cells, including type A cells, macrophage lineage cells and type B cells, which are specialized synovial fibroblasts. It is now widely recognized that synovial tissues are involved primarily in the pathogenesis of arthritic joint disorders by producing matrix-degenerating enzymes and proinflammatory cytokines. Accumulated evidence suggest that TGF-beta super family members play an essential role in bone and cartilage development. Wozney and co-workers (Wozney, J. M. (1989) Prog. Growth factor Res. 1:267-280) reported that bone morphogenetic proteins (BMPs) induce early cartilage formation. Recently it has been shown that ALK3 signaling that mediates BMP action has both stimulatory and regulatory roles in chondrogenesis to induce the chondrogenic differentiation of synovial fibroblastic cells (Seto et al (2004) J. Clin. Invest. 113:718-726). TDFRP compounds have been examined in chondrogenesis ex vivo and cell culture assays that may have relevance in osteoarthritis applications, cartilage repair, protection, and/or homeostasis.

TDFRP compounds can be used in bone tissue engineering. Lu et al., (Biochem Biophys Res Commun. 2003 Jun. 13; 305(4):882-90 have shown the efficacy of a BMP-polymer matrix in inducing the expression of the osteoblastic phenotype by muscle-derived cells and present a new paradigm for bone tissue engineering. TDFRP compounds may be used in bone transplantation (Rees and Haddad, Hosp Med. 2003 April; 64(4):205-9). TDFRP can also be used to promote bone healing. Maniscalco et al., (Acta Biomed Ateneo Parmense. 2002; 73(1-2):27-33) verify the therapeutic potential of this BMP-7 protein in fresh tibial closed fractures, using BMP-7 associated with osteosynthesis by means of a monolateral external fixator. Moreover, TDFRP compounds can be used in the regeneration of bone tissue, e.g., reconstructive surgery of the hip. Cook et al., (J Arthroplasty. 2001 December; 16(8 Suppl 1):88-94) demonstrated that the use of BMP-7 in conjunction with morcellized cancellous bone and cortical strut allograft in preclinical models dramatically improved the biologic activity of the graft, resulting in greater and earlier new bone formation and graft incorporation. The clinical use of BMP-7 in hip reconstructive procedures also resulted in greater and earlier new bone formation in the more challenging biologic environment compared with allograft bone alone.

TDFRP compounds can be used to treat skeletal defects e.g., acquired and congenital skeletal defects arise from trauma and developmental abnormalities as well as ablative cancer surgery. Rutherford et al., (Drug News Perspect. 2003 January-February; 16(1):5-10) discusses recent advances in bone morphogenetic protein 7 ex vivo gene therapy for localized skeletal regeneration address these limitations.

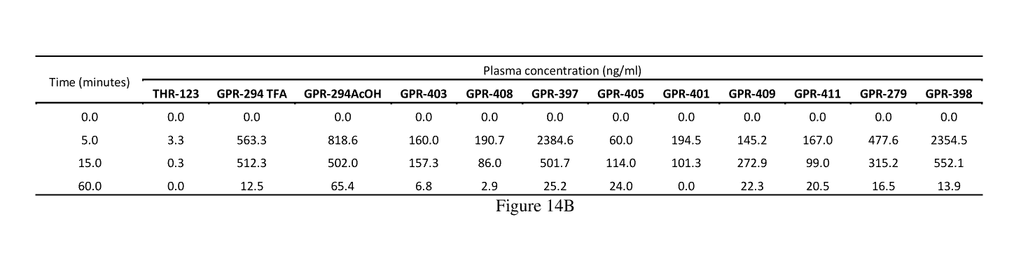

TDFRP compounds can be used in the prophylaxis or treatment of disorders of haematopoiesis. Studies by Detmer and Walker (Cytokine. 2002 Jan. 7; 17(1):36-42) indicate that individual BMPs form part of the complement of cytokines regulating the development of haematopoietic progenitors, and in particular, point to a role for BMP-4 in the control of definitive, as well as embryonic erythropoiesis. TDFRP compounds have been demonstrated to modulate cytokine production in various cell populations (e.g. HK-2 cell lines, cardiomyocytes, kidney tissues), repair kidney damage and/or protect kidneys from injury, which may affect the regulation and production of hematopoietic progenitor cells and growth factors (FIG. 14). TDFRP compounds can be used in the treatment of reproductive disorders, e.g., sterility. Zhao et al., (Dev Biol. 2001 Dec. 1; 240(1):212-22) demonstrated that mutation in BMP-7 exacerbates the phenotype of BMP-8a mutants in spermatogenesis and epididymis. These indicate that, similar to BMP-8a, BMP-7 plays a role in both the maintenance of spermatogenesis and epididymal function and it further suggests that BMP-8 and BMP-7 signal through the same or similar receptors in these two systems.

The present invention provides both prophylactic and therapeutic methods of treating a subject having a disease or disorder associated with aberrant TDF polypeptide or TDFRP compound target molecule expression or activity, or a method of delaying the progression of a disorder associated with aberrant TDF polypeptide or TDFRP compound target molecule expression or activity.

In one aspect, the present invention features methods of treating a subject having a disease or disorder associated with aberrant TDF polypeptide or TDFRP compound target molecule expression or activity, or a method of delaying the progression of a disorder associated with aberrant TDF polypeptide or TDFRP compound target molecule expression or activity, comprising administering to the subject an effective amount of a peptide comprising one or more of SEQ ID NO:1-72, thereby treating the disorder associated with aberrant TDF polypeptide or TDFRP compound target molecule expression or activity, or preventing the progression of the disorder associated with aberrant TDF polypeptide or TDFRP compound target molecule expression or activity, in the subject.

As discussed above, the disease or disorder may be a tissue degenerative disease, for example, but not limited to renal disease, heart disease, traumatic brain injury, stroke, atherosclerosis, arthritis, emphysema, osteoporosis, cardiomyopathy, cirrhosis, degenerative nerve disease, Holt-Oram disease, congenital disease, pulmonary disease, eye disease, diabetic nephropathy, degenerative bone disease, bone disorders, periodontal disease, chronic kidney disease, diabetes, cardiovascular disease, inflammatory disease, immune disease, skeletal disease, reproductive disease, hematopoetic disease, healing disorders, and cancer.

As also discussed above, the disease or disorder may be treated by the regeneration of tissues, organs or limbs, for example the regeneration of tissue selected from, but not limited to, muscle, bone, skin, epithelial, heart, nerve, endocrine, vessel, cartilage, periodontal, liver, retinal, and connective tissue.

It is understood and herein contemplated that the disclosed methods of treating a tissue degenerative disease can be combined with any other method of treating a tissue degenerative disease known in the art. It should be understood that a peptide, or a composition comprising a peptide comprising one or more of SEQ ID NO:1-72 of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the peptides of the present invention.

The combination therapy can include one or more peptides, e.g. peptides comprising one or more of SEQ ID NO:1-72, formulated with, and/or co-administered with, one or more additional therapeutic agents.

Kidney Diseases

In one embodiment, the disease or disorder associated with aberrant TDF polypeptide or TDFRP compound target molecule expression or activity is a kidney disease. Thus, the present invention also provides for both prophylactic and therapeutic methods of treating a subject at risk of (or susceptible to) a kidney disease or disorder.

Kidney diseases include renal dysfunction, disease and injury, e.g., ureteral obstruction, acute and chronic renal failure, chronic kidney disease, acute kidney injury, renal fibrosis, and diabetic nephropathy. Kidney diseases treated by the peptides of the invention may be atherosclerosis-related, hypertension-related, diabetes and/or autoimmune diseases-related.

In certain embodiments, the invention may be used to treat chronic kidney disease (CKD). Chronic kidney disease (CKD), also known as chronic renal disease, is a progressive loss in renal function over a period of months or years. The symptoms of worsening kidney function are non-specific, and chronic kidney disease is often diagnosed as a result of screening of people known to be at risk of kidney problems.

Chronic kidney disease may be identified by a blood test, for example for creatinine. Higher levels of creatinine indicate a lower glomerular filtration rate and as a result a decreased capability of the kidneys to excrete waste products. CKD has been classified into 5 stages, where stage 1 is kidney damage with normal GFR (mL/min/1.73 m²) of .gtoreq.90; stage 2 is kidney damage with a mild decrease in GFR (GFR 60-89); stage 3 is a moderate decrease in GFR (GFR 30-59); stage 4 is a severe decrease in GFR (GFR 15-29); and stage 5 is kidney failure (GFR<15 or dialysis). Stage 5 CKD is often called End Stage Renal Disease (ESRD) and is synonymous with the now outdated terms chronic kidney failure (CKF) or chronic renal failure (CRF).

Accordingly, in one aspect, the present invention relates to methods of delaying the progression of a kidney disease or disorder in a subject, for example the progression from stage 1 chronic kidney disease to stage 2 chronic kidney disease, for example the progression from stage 2 chronic kidney disease to stage 3 chronic kidney disease, for example the progression from stage 3 chronic kidney disease to stage 4 chronic kidney disease, for example the progression from stage 4 chronic kidney disease to stage 5 chronic kidney disease. The subject with chronic kidney disease according to the present invention may be a subject with stage 1 chronic kidney disease, stage 2 chronic kidney disease, stage 3 chronic kidney disease, stage 4 chronic kidney disease, or stage 5 chronic kidney disease.

Kidney disease may also be identified by determining the expression level of a gene in a subject suspected of having the disease. For example, the present application has identified that higher levels of IL-6, KIM1, NGAL, clusterin, osteopntin, and/or vimentin are associated with increased proteinuria. Therefore, kidney disease may be identified by determining the expression level of a gene in a subject suspected of having the disease and comparing the expression level of the gene to a control level of the gene in a healthy subject, thereby identifying that the subject has kidney disease.

Conversely, a subjects response to treatment with a peptide of the invention may be monitored by determining the expression level of a gene in the subject suspected receiving treatment with the peptide. For example, the present application has identified that higher levels of IL-6, KIM1, NGAL, clusterin, osteopntin, and/or vimentin are associated with increased proteinuria, and that treatment with peptides of the invention lead to a decrease in the expression levels of these genes. Therefore, response to treatment with a peptide of the invention may be identified by determining the expression level of a gene in a subject receiving treatment with a peptide of the invention and comparing the expression level of the gene to a control level of the gene in a healthy subject, thereby identifying that the subject is responding to treatment with the peptide. Alternatively, response to treatment with a peptide of the invention may be identified by determining the expression level of a gene in a subject receiving treatment with a peptide of the invention and comparing the expression level of the gene to a control level of the gene in the subject taken from before the subject received treatment with a peptide of the invention, thereby identifying that the subject is responding to treatment with the peptide.

In other certain embodiments, the invention may be used to treat acute kidney disease.

In one aspect, the invention features methods of treating a subject having a kidney disease or disorder, comprising administering to the subject an effective amount of a peptide comprising one or more of SEQ ID NO:1-72, thereby treating the kidney disease or disorder in the subject. In certain embodiments, the peptide consists of the amino acid sequence of SEQ ID NO:1-72. In further embodiments, the peptide has at least 50% identity to SEQ ID NO:1-72. In one embodiment, the peptide comprises SEQ ID NO:25. In another embodiment, the peptide consists of SEQ ID NO:25.

It is understood and herein contemplated that the disclosed methods of treating a kidney disease or disorder can be combined with any other method of treating a kidney disease or disorder known in the art.

In certain embodiments, the peptides disclosed herein can be compounds used to treat renal dysfunction, disease and injury, e.g. ureteral obstruction, acute and chronic renal failure, acute kidney disease, renal fibrosis, and diabetic nephropathy. Specifically, the peptides of the invention can be used to treat kidney disease, e.g., chronic kidney disease.

In certain other embodiments, the invention may be used to treat CKD. Chronic kidney disease (CKD) is a disease afflicting an estimated 13% of Americans. Regardless of disease origin, fibrosis is a final common pathway in CKD that leads to disease progression and ultimately organ failure. Chronic kidney disease is progressive, not curable, and ultimately fatal, either because of the consequences of kidney failure or due to the high level of cardiovascular mortality in the CKD patient population.

In certain other embodiments, the peptides can be used in the prophylaxis or treatment of renal fibrosis and CKD.

Thus, in various aspects, the invention includes methods of administering the peptides disclosed herein for therapeutic purposes for any kidney disease or disorder. The peptides of the invention can be administered in vitro (e.g., by culturing the cell with the peptide) or, alternatively, in vivo (e.g., by administering the peptide to a subject or by administering a somatic gene transfer vector which then expresses the peptide in the subject as means for administration of the peptide). As such, the invention provides methods of treating an individual afflicted with a kidney disease or disorder. Effective dosages and schedules for administering the compositions of the invention may be determined empirically, and making such determinations is within the skill in the art. The dosage ranges for the administration of the compositions are those large enough to produce the desired effect in which the symptoms/disorder are/is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, in certain embodiments, a dosage of 0.0001 to 10,000 mg/kg body weight is administered to the subject per day. In other embodiments, the administered dosage is from 1 to 100 mg/kg body weight per day. Different dosing regimens may be used as appropriate.

According to certain embodiments of the present invention, the peptide is administered to the subject orally. The peptides of the invention may also be administered to the subject topically, enterally, or parenterally. In certain embodiments, a peptide of the invention is administered to a subject that has been identified as having CKD or is selected based on having CKD.

Following administration of a disclosed composition, such as a peptide of the invention, for treating, inhibiting, or preventing kidney disease or disorder, the efficacy of the therapeutic peptide can be assessed in various ways well known to the skilled practitioner.

The compositions disclosed herein may be administered prophylactically to patients or subjects who are at risk for a kidney disease or disorder.

Kits and/or Pharmaceutical Packages

The present invention also contemplates kits and pharmaceutical packages that are drawn to reagents or components that can be used in practicing the methods disclosed herein. The kits can include any material or combination of materials discussed herein or that would be understood to be required or beneficial in the practice of the disclosed methods. For example, the kits could include a peptide of the invention, or one or more additional active agents. In addition, a kit can include a set of instructions for using the components of the kit for its therapeutic and/or diagnostic purposes.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The following examples further demonstrate several embodiments of this invention. While the examples illustrate the invention, they are not intended to limit it.

### EXAMPLES

The structures, materials, compositions, and methods described herein are intended to be representative examples of the invention, and it will be understood that the scope of the invention is not limited by the scope of the examples. Those skilled in the art will recognize that the invention may be practiced with variations on the disclosed structures, materials, compositions and methods, and such variations are regarded as within the ambit of the invention.

### Example 1

### Identification of Metabolites of THR-123

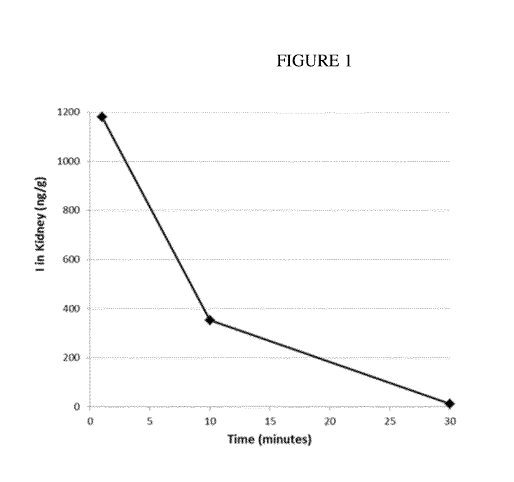

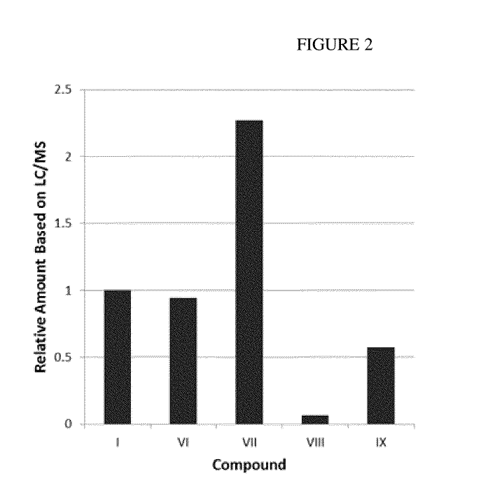

THR-123 was found to have beneficial effect to treat kidney disease (see, e.g. U.S. Pat. No. 7,482,329, U.S. Pat. No. 8,455,446, both of which are incorporated by reference in their entireties herein). When administered intravenously to rodents, the peptide has a short half-life in plasma and kidney tissue (FIG. 1). Ten minutes post administration of an i.v. administration of THR-123, the metabolites VI, VII, VIII and IX, in addition to THR-123 were identified in the kidney. FIG. 2 shows the relative amount of metabolites of THR-123 in rat kidney tissue 10 minutes post IV administration. The main metabolite VII results from a proteolytic cleavage between positions 14 and 15 (VII in FIG. 2). The structure of each metabolite was confirmed by synthesis and analytical characterization. The structures are shown in table 2, below.

TABLE 2
THR-123 metabolites

| | Position | | | | | | | | | | | | | | | |
|---|---|---|---|---|---|---|---|---|---|---|---|---|---|---|---|---|
| Compound | 1 | 2 | 3 | 4 | 5 | 6 | 7 | 8 | 9 | 10 | 11 | 12 | 13 | 14 | 15 | 16 |
| I (THR-123) | C | Y | F | D | D | S | S | N | V | L | C | K | K | Y | R | S |
| VI (GPR-263) | C | Y | F | D | D | S | S | N | V | L | C | | | | | |
| VII (GPR-250) | C | Y | F | D | D | S | S | N | V | L | C | K | K | Y | | |
| VIII (GPR-262) | C | Y | F | D | D | S | S | N | V | L | C | K | | | | |
| IX (GPR-261) | C | Y | F | D | D | S | S | N | V | L | C | K | K | | | |

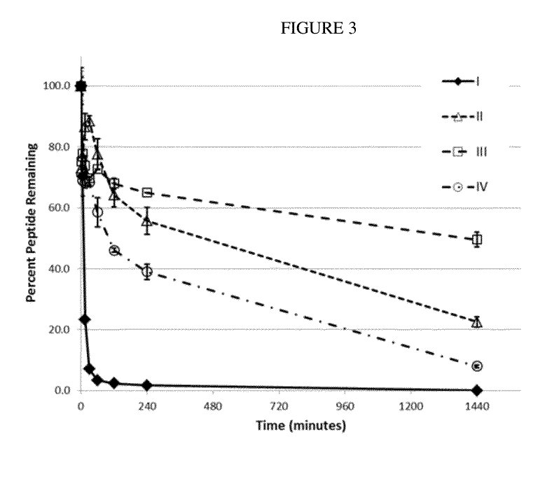

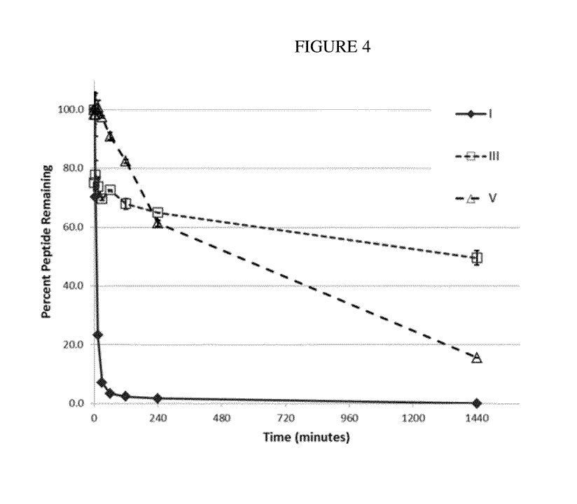

The present invention has found that the stability of compound toward plasma proteolytic cleavage could be enhanced (table 3) by incorporating a D amino acid in position 13, (Compound II) 14 (Compound III) and 15 (Compound IV) or N-Me amino acid in position 15 (Compound V) (FIGS. 3 and 4).

TABLE 3
Peptides structures used to explore the plasma stability of THR-123

| | Position | | | | | | | | | | | | | | | |
|---|---|---|---|---|---|---|---|---|---|---|---|---|---|---|---|---|
| Compound | 1 | 2 | 3 | 4 | 5 | 6 | 7 | 8 | 9 | 10 | 11 | 12 | 13 | 14 | 15 | 16 |
| I (THR-123) | C | Y | F | D | D | S | S | N | V | L | C | K | K | Y | R | S |
| II (GPR-388) | C | Y | F | D | D | S | S | N | V | L | C | K | k* | Y | R | S |
| III (GPR-294) | C | Y | F | D | D | S | S | N | V | L | C | K | K | y* | R | S |
| IV (GPR-389) | C | Y | F | D | D | S | S | N | V | L | C | K | K | Y | r* | S |
| V (GPR_396) | C | Y | F | D | D | S | S | N | V | L | C | K | K | Y | Me-R* | S |

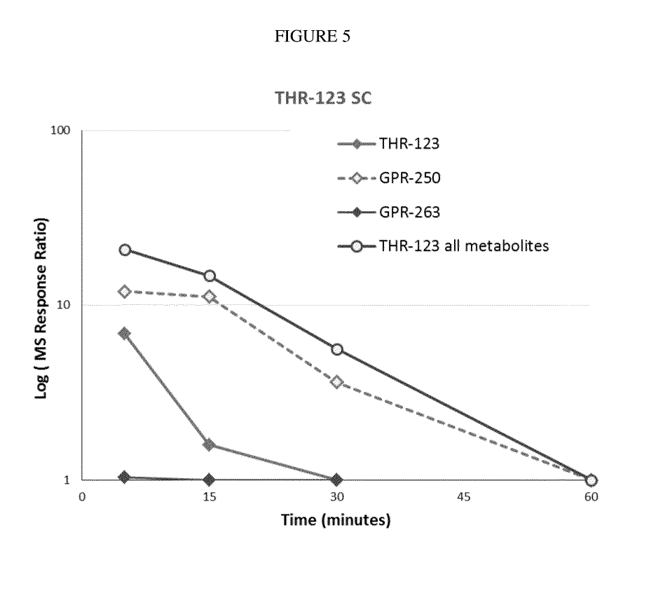

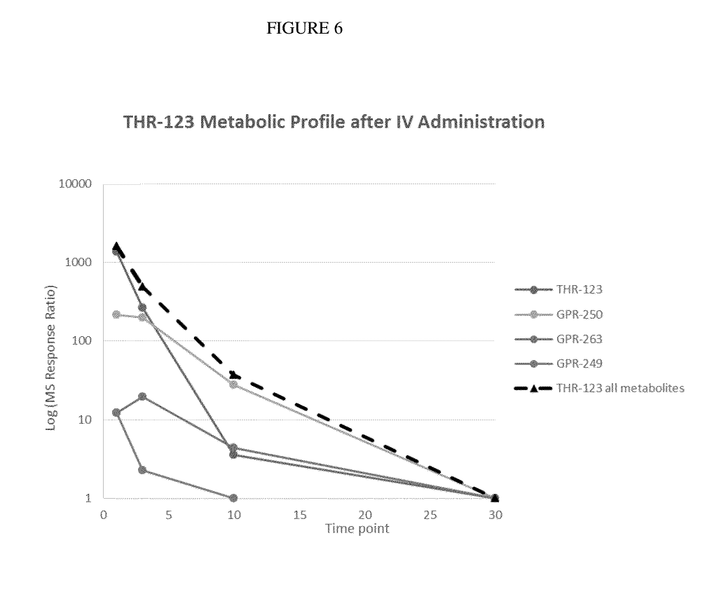

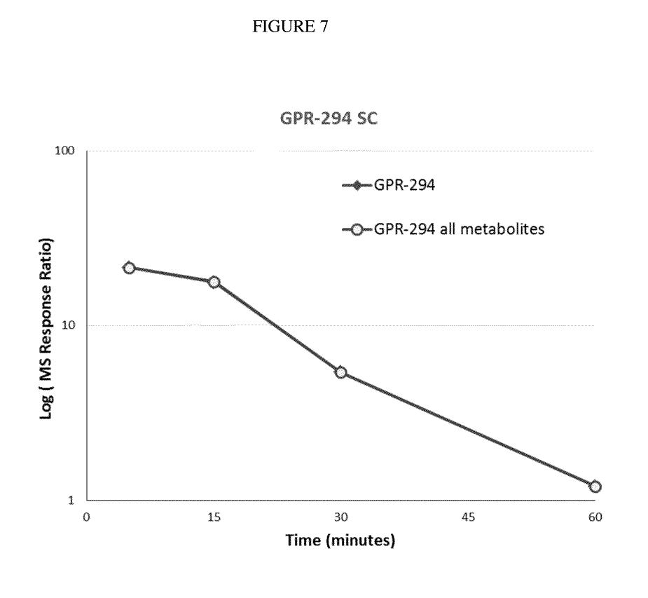

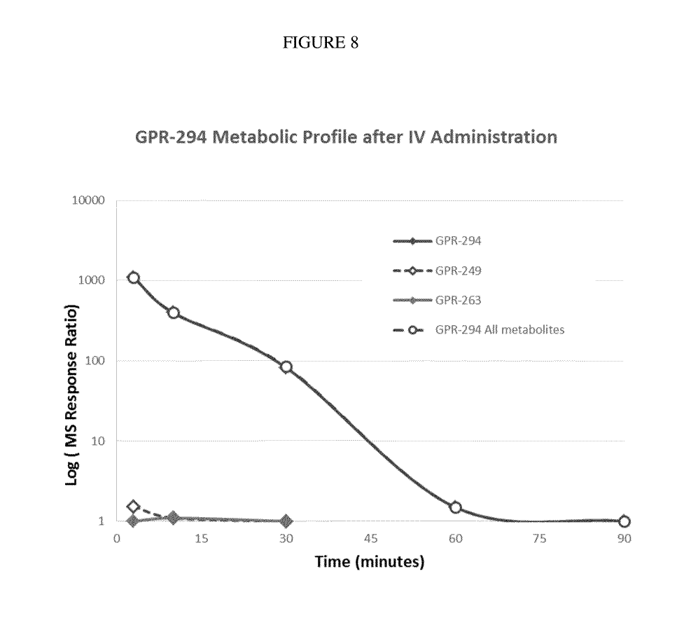

Further work demonstrated that, unexpectedly, the incorporation of the D amino acid at position 14 not only prevented the proteolytic cleavage between the amino acids 14 and 15, but also reduced drastically the cleavage at all the other potential proteolytic site as shown in FIGS. 5, 6, 7, and 8.

### Example 2

### Synthesis of the Peptides

The peptides were prepared using standard Fmoc solid phase synthesis on Wang resin. A person skilled in the art will understand that there are other options for the synthesis of the peptides from this invention such as liquid phase synthesis. Solid phase synthesis will be the preferred approach due to the presence of unnatural amino acid thus making recombinant methods less useful. Fragment based approaches such as using one part recombinant combined with liquid phase coupling may be useful for larger scale syntheses.

The syntheses used to prepare examples of this invention were initiated on HO-Wang or Fmoc-Ser(tBu)-Wang resins with a 0.5-0.6 mmol/g substitution level. The side chain protections on the Fmoc amino acids were: tert-butyl (tBU) on Asp, Tyr, Ser; trityl (Trt) on Asn, Cys; tert-butyloxycarbonyl (Boc) on Lys; pentamethyl-2,3-dihydrobenzofuran-5-sulfonyl (Pbf) on Arg.

Synthesis Summary:

The Fmoc group was removed using 20% piperidine in DMF or 20% piperidine and 0.1% HOBt in NMP, 20 min. This method was used for each Fmoc deprotection. All acylation reactions were carried out using 3-fold of Fmoc amino acids activated with DIC (3 eq.) in the presence of HOBt (3 eq.). Each coupling reaction took on average 2-3 h for completion. Kaiser test was used throughout for the in-process control of the coupling completion and the Fmoc deprotection. Re-coupling was repeated when the result of Kaiser Test was not satisfactory. Peptidyl resin was extensively washed with DCM and methanol to remove the trace of DMF and dried at RT overnight. The optimization of cleavage condition was performed on small samples of peptidyl resins. The peptidyl resin was stirred in a mixture of TFA/EDT/H₂O/TIS or Reagent K for 3 h under nitrogen bubbling to cleave the linear peptide from the resin. Crude linear peptides were collected by precipitation in cold diethyl ether. The disulfide bond formation was carried out with iodine in acetic acid solution (The dry crude peptide solid was completely dissolved in 20% acetic acid to a concentration of ˜2 mg/mL. Under stirring, a 5% iodine/methanol solution was added drop-wise until a persistent yellow hue is obtained. The solution was stirred for another 15 min and then quenched with a solution of 0.1M thiosulfide which was added drop-wise until the yellow color disappears).

Purification Summary:

Method development was performed on a 4.6×250 mm analytical column to ascertain an appropriate gradient system for scale-up. The crude peptide was purified with Waters LC 4000 prep system (Flow rate up to 300 ml/min) equipped with Phenomenex Gemini C-18 prep column. The fractions containing the desired peptide with the appropriate purity were combined, frozen and lyophilized (Savant SuperModulyo (Fisher Thermo) until complete dryness.

Analysis Summary:

The peptides were analyzed by HPLC by dual wavelength detection and MALDI-TOF mass spectrometry. (HPLC: Waters 600 Multi-solvent delivery system HPLC equipped with a 2486 dual wavelength detector and a 717 plus autosampler, MS: Applied Biosystems Voyager DE).

Compound I (THR-123)

(Seq. ID. No. 1)
Cys-Tyr-Phe-Asp-Asp-Ser-Ser-Asn-Val-Leu-Cys-Lys-
Lys-Tyr-Arg-Ser

The Fmoc amino acids Fmoc-(Pdf)Arg, Fmoc-(tBu)Tyr, Fmoc-(ε-Boc)Lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1924.8 Da, theoretical: 1926.1339 Da).

Compound II (GPR-388)

(Seq. ID. No. 2)
Cys-Tyr-Phe-Asp-Asp-Ser-Ser-Asn-Val-Leu-Cys-Lys-
(D)lys-Tyr-Arg-Ser

The Fmoc amino acids Fmoc-(Pdf)Arg, Fmoc-(tBu)Tyr, Fmoc-(ε-Boc)-D-lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1926.0210 Da (average mass), theoretical: 1926.1339 Da).

Compound III (GPR-294)

(Seq. ID. No. 3)
Cys-Tyr-Phe-Asp-Asp-Ser-Ser-Asn-Val-Leu-Cys-Lys-
Lys-D-tyr-Arg-Ser

The Fmoc amino acids Fmoc-(Pdf)Arg, Fmoc-(tBu)-D-tyr, Fmoc-(ε-Boc)Lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1924.9812 Da, theoretical: 1926.1339 Da).

Compound IV (GPR-389)

(Seq. ID. No. 4)
Cys-Tyr-Phe-Asp-Asp-Ser-Ser-Asn-Val-Leu-Cys-Lys-
Lys-Tyr-D-arg-Ser

The Fmoc amino acids Fmoc-(Pdf)-D-arg, Fmoc-(tBu)Tyr, Fmoc-(ε-Boc)Lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1925.9544 Da, theoretical: 1926.1339 Da).

Compound V (GPR-396)

(Seq. ID. No. 5)
Cys-Tyr-Phe-Asp-Asp-Ser-Ser-Asn-Val-Leu-Cys-Lys-
Lys-Tyr-(Na-Me)Arg-Ser

The Fmoc amino acids Fmoc-(Nω-Pdf)-(Nα-Me)Arg, Fmoc-(tBu)Tyr, Fmoc-(ε-Boc)Lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1938.8916 Da, theoretical: 1938.8604 Da).

Compound VI (GPR-263)

(Seq. ID. No. 6)
Cys-Tyr-Phe-Asp-Asp-Ser-Ser-Asn-Val-Leu-Cys

The Fmoc amino acids Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Wang resin and cyclized using the above synthetic methodologies. The desired peptide was purified as a TFA salt to >95% by HPLC and lyophilized (MS 1262.4788 Da, theoretical: 1262.4584 Da).

Compound VII (GPR-250)

(Seq. ID. No. 7)
Cys-Tyr-Phe-Asp-Asp-Ser-Ser-Asn-Val-Leu-Cys-Lys-
Lys-Tyr

The Fmoc amino acids Fmoc-(tBu)Tyr, Fmoc-(ε-Boc)Lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Wang resin and cyclized using the above synthetic methodologies. The desired peptide was purified as a TFA salt to >95% by HPLC and lyophilized (MS 1681.7184 Da, theoretical: 1681.7116 Da).

Compound VIII (GPR-262)

(Seq. ID. No. 8)
Cys-Tyr-Phe-Asp-Asp-Ser-Ser-Asn-Val-Leu-Cys-Lys

The Fmoc amino acids Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Wang resin and cyclized using the above synthetic methodologies. The desired peptide was purified as a TFA salt to >95% by HPLC and lyophilized (MS 1390.5940 Da, theoretical: 1390.5534 Da).

Compound IX (GPR-261)

(Seq. ID. No. 9)
Cys-Tyr-Phe-Asp-Asp-Ser-Ser-Asn-Val-Leu-Cys-Lys-
Lys

The Fmoc amino acids Fmoc-(ε-Boc)Lys, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Wang resin and cyclized using the above synthetic methodologies. The desired peptide was purified as a TFA salt to >95% by HPLC and lyophilized (MS 1518.7758 Da, theoretical: 1518.6483 Da).

Compound X (GPR-293)

(Seq. ID. No. 10)
Cys-Tyr-Phe-Asp-Asp-Ser-Ser-Asn-Val-Leu-Cys-Lys-
Arg-Tyr-Arg-Ser

The Fmoc amino acids Fmoc-(Pdf)Arg, Fmoc-(tBu)Tyr, Fmoc-(Pdf)Arg, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS M+H 1954.95 Da, theoretical: 1953.8519 Da, monoisotopic 1952.8509 Da).

Compound XI (GPR-397)

(Seq. ID. No. 11)
Cys-Tyr-Phe-Asp-Asp-Ser-Ser-Asn-Val-Leu-Cys-Lys-
Arg-D-tyr-Arg-Ser

The Fmoc amino acids Fmoc-(Pdf)Arg, Fmoc-(tBu)-D-tyr, Fmoc-(Pdf)Arg, Fmoc-(ε-Boc)Lys, Fmoc-(Trt)Cys, Fmoc-Leu, Fmoc-Val, Fmoc-(Trt)Asn, Fmoc-(tBu)Ser, Fmoc-(tBu)Ser, Fmoc-(tBu)Asp, Fmoc-(tBu)Asp, Fmoc-Phe, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1953.0060 Da, theoretical: 1952.8509 Da).

Compound XII (GPR-267)

(Seq. ID. No. 12)
Cys-Tyr-Tyr-Asp-Asp-Ser-Ser-Ser-Val-Leu-Cys-Lys-
Lys-Tyr-Arg-Ser

The Fmoc amino acids Fmoc-(Pdf)Arg, Fmoc-(tBu)-Tyr, Fmoc-(ε-Boc)Lys, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(tBu)Asp, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS M+H 1914.7 Da, theoretical: 1914.8361 Da).

Compound XIII (GPR-295)

(Seq. ID. No. 13)
Cys-Tyr-Tyr-Asp-Asp-Ser-Ser-Ser-Val-Leu-Cys-Lys-
Lys-D-tyr-Arg-Ser

The Fmoc amino acids Fmoc-(Pdf)Arg, Fmoc-(tBu)-D-tyr, Fmoc-(ε-Boc)Lys, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(tBu)Asp, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS M+H 1915.51 Da, theoretical: 1914.8306 Da).

Compound XIV(GPR-269)

(Seq. ID. No. 14)
Cys-Tyr-Tyr-Asp-Asn-Ser-Ser-Ser-Val-Leu-Cys-Lys-
Lys-Tyr-Arg-Ser

The Fmoc amino acids Fmoc-(Pdf)Arg, Fmoc-(tBu)-Tyr, Fmoc-(ε-Boc)Lys, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(Trt)Asn, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1913.8980 Da, theoretical: 1912.8448 Da).

Compound XV(GPR-296)

(Seq. ID. No. 15)
Cys-Tyr-Tyr-Asp-Asn-Ser-Ser-Ser-Val-Leu-Cys-Lys-
Lys-D-tyr-Arg-Ser

The Fmoc amino acids Fmoc-(Pdf)Arg, Fmoc-(tBu)-D-tyr, Fmoc-(ε-Boc)Lys, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(Trt)Asn, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS 1914.53 Da, theoretical: 1913.8520 Da, monoisotopic 1912.8448 Da).

Compound XVI (GPR-259)

(Seq. ID. No. 16)
Cys-Tyr-Tyr-Asp-Asn-Ser-Ser-Ser-Val-Leu-Cys-Lys-
Arg-Tyr-Arg-Ser

The Fmoc amino acids Fmoc-(Pdf)Arg, Fmoc-(tBu)-Tyr, Fmoc-(Pdf)Arg, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(Trt)Asn, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS Da, theoretical: 1940.8509 Da).

Compound XVII (GPR-279)

(Seq. ID. No. 17)
Cys-Tyr-Tyr-Asp-Asn-Ser-Ser-Ser-Val-Leu-Cys-Lys-
Arg-D-tyr-Arg-Ser

The Fmoc amino acids Fmoc-(Pdf)Arg, Fmoc-(tBu)-D-tyr, Fmoc-(Pdf)Arg, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(Trt)Asn, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS Da, theoretical: 1940.8509 Da).

Compound XVIII (GPR-398)

(Seq. ID. No. 18)
Cys-Tyr-Tyr-Asp-Asn-Ser-Ser-Ser-Val-Leu-Cys-Lys-
Arg-Tyr-(Nα-Me)Arg-Ser

The Fmoc amino acids Fmoc-(Pdf)Arg, Fmoc-(tBu)-Tyr, Fmoc-(Nω-Pdf)-(Nα-Me)Arg, Fmoc-(ε-Boc)Lys, Fmoc-(Trt)Cys, Fmoc-Leu, Fmoc-Val, Fmoc-(tBu)Ser, Fmoc-(tBu)Ser, Fmoc-(tBu)Ser, Fmoc-(Trt)Asn, Fmoc-(tBu)Asp, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS Da, theoretical: 1954.8666 Da).

Compound XVIII (GPR-405)

(Seq. ID. No. 25)
Cys-Tyr-Tyr-Phe-Asn-Ser-Ser-Gln-Val-Leu-Cys-Lys-
Arg-(D)Tyr-Arg-Ser

The Fmoc amino acids Fmoc-(Pdf)Arg, Fmoc-(tBu)-D-tyr, Fmoc-(Nω-Pdf)-Arg, Fmoc-(ε-Boc)Lys, Fmoc-(Trt)Cys, Fmoc-Leu, Fmoc-Val, Fmoc-(Trt)Gln, Fmoc-(tBu)Ser, Fmoc-(tBu)Ser, Fmoc-(Trt)Asn, Fmoc-Phe, Fmoc-(tBu)-Tyr, Fmoc-(tBu)Tyr and Fmoc-(Trt)Cys were sequentially attached to the Fmoc-Ser(tBu)-Wang resin and cyclized using the above synthetic methodologies. The desired peptide was purified as a TFA salt to >95% by HPLC and lyophilized (MS Da, theoretical: 2015.2779 Da, measured: 2015.047 Da).

### Example 3

### In-Vitro Incubations

GPR-397 and GPR-405 were independently added to fresh rat plasma at a final in-vial concentration of 1 μM. The peptides were incubated in a water bath at 37° C. over a time course that included the following points (in minutes): 0, 1, 5, 15, 30, 60, 120, 240, and 1440. After the incubation time was completed, a 50 μL aliquot of plasma was removed and proteins were precipitated using a solution containing internal standard (THR-123 at 150 ng/mL) in methanol with 0.5% acetic acid (v/v). The samples were centrifuged at 13,000 RPM for 5 minutes, and a 100 μL aliquot of supernatant was removed and transferred to a 96-well plate containing 100 μL of reconstitution solution (Water/MeOH/Acetic Acid/TFA, 60/40/0.2/0.05, v/v/v/v). A calibration curve and quality control (QC) samples were prepared in parallel in the same manner as the incubation samples. The calibration range for this assay was from 0.02 to 2.0 μM in rat plasma. The extracted calibrants, QC, and incubation samples were injected onto an AB SCIEX API 2000 LC-MS/MS system. Chromatographic separation was performed using a reversed-phase C18 HPLC column (Ace-3 C18, 30×2.1 mm, 3 μm). The solvent system consisted of Milli-Q water, acetonitrile and formic acid. Analyte and internal standard were detected using the multiple reaction monitoring (MRM) scan mode. Calibration curves were generated by using a weighted (1/concentration) quadratic regression analysis of the peak area ratios (analyte/internal standard) versus the nominal concentration of the calibration standards. The concentration of the analyte in the incubation samples was determined by back-calculating the peak area ratios obtained using the quadratic regression.

Results from the in vitro plasma stability studies for THR-123 and GPR-405 are presented in Tables 4 and 5 below.

TABLE 4
Plasma concentrations of GPR-405 over time in vitro

| Time | Sample 1 | Sample 2 | Mean | SD | TI remaining |
|---|---|---|---|---|---|
| (min) | (μM) | (μM) | (μM) | (μM) | (%) |
| 0 | 1.100 | 1.090 | 1.095 | 0.007 | 100 |
| 1 | 1.080 | 1.050 | 1.065 | 0.021 | 97.3 |
| 5 | 1.090 | 1.120 | 1.105 | 0.021 | 100.9 |
| 15 | 1.080 | 1.050 | 1.065 | 0.021 | 97.3 |
| 30 | 0.963 | 1.040 | 1.002 | 0.054 | 91.5 |
| 60 | 0.895 | 0.973 | 0.934 | 0.055 | 85.3 |
| 120 | 0.836 | 0.815 | 0.826 | 0.015 | 75.4 |
| 240 | 0.534 | 0.541 | 0.538 | 0.005 | 49.1 |
| 1440 | 0.081 | 0.068 | 0.075 | 0.009 | 6.8 |

| | |
|---|---|
| AUC0-240 (μM* min/ml) | 195.4 |

GPR-405 exhibits 97.3% of the parent compound remaining after 15 minutes, after which it shows degradation over time with 49.1% remaining after 4 hours and 6.8% remaining after 24 hours.

TABLE 5
Plasma concentrations of THR-123 over time in vitro

| Time | Sample 1 | Sample 2 | Mean | SD | TI remaining |
|---|---|---|---|---|---|
| (min) | (μM) | (μM) | (μM) | (μM) | (%) |
| 0 | 0.970 | 0.840 | 0.905 | 0.092 | 100 |
| 1 | 0.850 | 0.860 | 0.855 | 0.007 | 94.4 |
| 5 | 0.300 | 0.290 | 0.295 | 0.007 | 32.4 |
| 15 | 0.060 | 0.050 | 0.055 | 0.007 | 6.4 |
| 30 | 0.060 | 0.030 | 0.030 | 0.000 | 3.3 |
| 60 | 0.026 | 0.024 | 0.025 | 0.001 | 2.8 |
| 120 | 0.015 | 0.020 | 0.018 | 0.004 | 1.9 |
| 240 | 0.010 | 0.011 | 0.011 | 0.001 | 1.2 |

| | |
|---|---|
| AUC0-240 (μM* min/ml) | 15.65 |

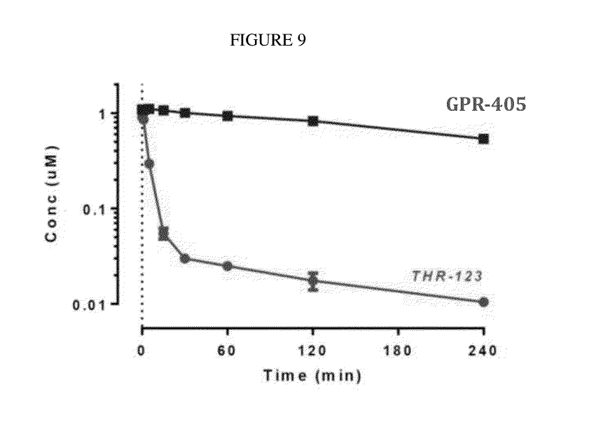

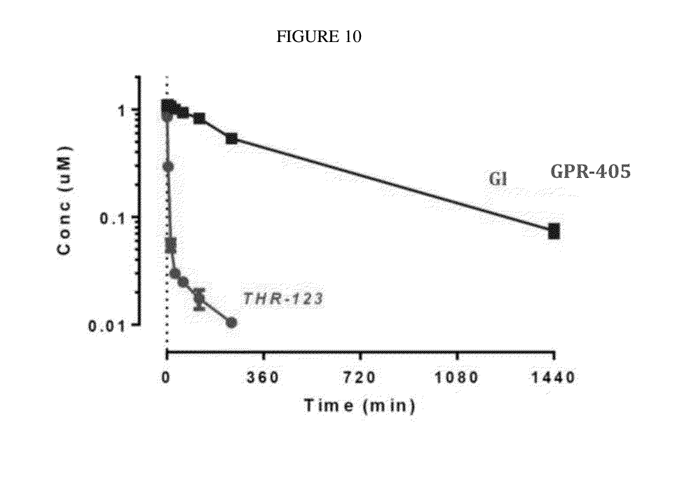

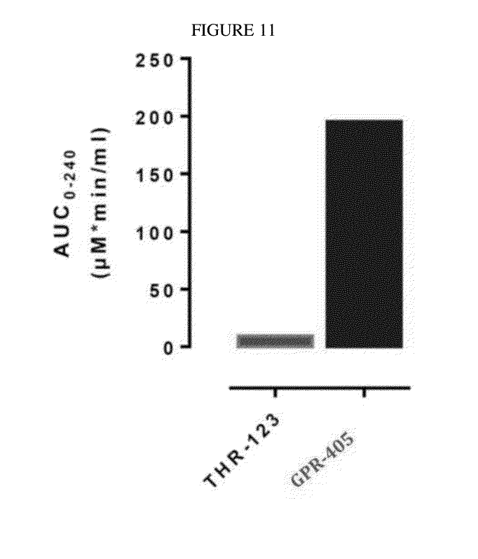

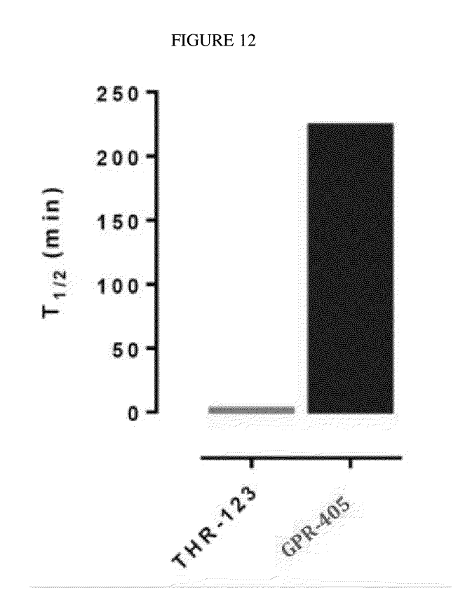

FIGS. 9-12 compare the in vitro plasma stability results for GPR-405 and THR-123 incubated in fresh rat plasma treated with heparin. FIG. 9 provides a scatter plot showing the first 240 minutes of the time-course and disappearance of the THR peptides in heparin-treated fresh rat plasma at 37° C. FIG. 10 shows the entire time course over 1440 minutes. FIG. 11 compares the area under the curve (AUC) between time 0 and 240 minutes for each peptide. FIG. 12 compares the calculated half-life of THR-123 and GPR-405. Overall, the results show that GPR-405 is dramatically more stable than THR-123 under the same conditions in fresh rat plasma treated with heparin in vitro at 37° C., with a measured half-life about 100 times greater than that of THR-123.

### Example 4

### In-Vivo Pharmacokinetic Studies

A stock solution of THR-123 was prepared at 250 μg/mL in DMSO. This solution was quantitatively added to blank mouse plasma to generate a calibration curve. The linear range was from 0.25 to 250 ng/mL. A 175 μL aliquot of plasma from removed from each calibrant tube and processed using solid phase extraction (SPE). Study samples were first diluted with blank control mouse plasma, and 175 μL was aliquoted for processing using SPE. An internal standard working solution (ISWS) was added to each calibrant and study sample (GPR-227 at 100 ng/mL in water). Plasma proteins were precipitated with 175 μL of methanol with 0.5% acetic acid (v/v). After centrifugation at 14,800 RPM for 10 minutes, a 300 μL aliquot of supernatant was diluted in water with 4% phosphoric acid (v/v). The entire diluted sample was extracted by SPE using a Waters Oasis HLB 96-well μElution plate. Samples were washed twice using a solution of acidified water (0.1% TFA, v/v) and 15% MeOH in water (v/v). Purified extracts were eluted into a 96-well collection plate using 40 μL of elution solvent. Standards and study samples were injected onto a Thermo Q-exactive LC-MS/MS system. Chromatographic separation was performed using a reversed-phase C18 HPLC column (Waters CSH C18, 50×2.1 mm, 2.5 μm). Analyte and internal standard were detected using the targeted selected ion monitoring (tSIM) scan mode. Calibration curves were generated by using a weighted (1/concentration²) linear regression of the peak area ratios (analyte/internal standard) versus the nominal concentration of the calibration standards. The concentration of the analyte in the study samples was determined by back-calculating the peak area ratios obtained using the linear regression and applying the appropriate dilution factors.

### Example 5

### Ex-Vivo Plasma Stability Protocol

Test article is added to fresh mouse or rat plasma at a final in-vial concentration of 1 μM. The peptide is incubated in a water bath at 37° C. over a time course that included the following points (in minutes): 0, 15, 30, 60, 120, 240, and 1440. After the incubation time is completed, a 50 μL aliquot of plasma is removed and proteins are precipitated using a solution containing internal standard (THR-123 at 150 ng/mL) in methanol with 0.5% acetic acid (v/v). The samples are centrifuged at 13,000 RPM for 5 minutes, and a 100 μL aliquot of supernatant is removed and transferred to a 96-well plate containing 200 μL of reconstitution solution (Water/MeOH/Acetic Acid/TFA, 60/40/0.2/0.05, v/v/v/v). A calibration curve and quality control (QC) samples are prepared in parallel in the same manner as the incubation samples. The calibration range for this assay is from 0.02 to 2.0 μM in rat or mouse plasma. The extracted calibrants, QC, and incubation samples are injected onto an AB SCIEX API 2000 LC-MS/MS system. Chromatographic separation is performed using a reversed-phase C18 HPLC column (Ace-3 C18, 30×2.1 mm, 3 μm). The solvent system consists of Milli-Q water, acetonitrile and formic acid. Analyte and internal standard are detected using the multiple reaction monitoring (MRM) scan mode. Calibration curves are generated by using a weighted (1/concentration) quadratic regression analysis of the peak area ratios (analyte/internal standard) versus the nominal concentration of the calibration standards. The concentration of the analyte in the incubation samples is determined by back-calculating the peak area ratios obtained using the quadratic regression.

Ex-vivo plasma stability studies have been completed for the following Compounds, and the results are shown in table 6, below.

Stabilized peptides: GPR-295, GPR-397, GPR-296, GPR-396, GPR-279, GPR-398, GPR-294, GPR-388, GPR-389

Non-stabilized peptides: GPR-269, GPR-259, THR-123, GPR-293, GPR-267

TABLE 6
Results of ex-vivo stability studies

Percent remaining

| Compound | 60 min | 120 min |
|---|---|---|
| GPR-295 | 92.2 | 94.7 |
| GPR-397 | 92.0 | 89.3 |
| GPR-296 | 97.5 | 86.4 |
| GPR-396 | 91.0 | 82.4 |
| GPR-279 | 89.1 | 77.8 |
| GPR-398 | 79.2 | 68.1 |
| GPR-294 | 72.6 | 67.9 |
| GPR-388 | 77.6 | 64.2 |
| GPR-389 | 58.5 | 46.1 |
| GPR-269 | 9.9 | 6.8 |
| GPR-259 | 4.0 | 2.5 |
| THR-123 | 3.4 | 2.3 |
| GPR-293 | 3.5 | 1.6 |
| GPR-267 | 1.5 | 0.0 |

### Example 6

### Pharmacokinetics' Procedures (PK)

The study described herein involved single (mouse) or multiple (rat) blood collections after test article administration. Test articles are given by oral (PO), intravenous (IV), subcutaneous (SC) or intraperitoneal (IP) routes. All animals have a five day acclimatization period before dosing. Body weights are recorded prior to dosing. For mouse PK, 3 animals per group per time point are used (terminal bleed). For rats, multiple blood samplings per animal (adequately spaced in time) are taken. At the end of the study, all animals are euthanized by CO2 and final blood sample was collected via cardiac puncture.

Dose Formulation:

Preparations are performed under a laminar flow hood using clean procedures. The formulations are kept at in a refrigerator set to maintain 4° C. before use. The dose volume for each animal is based on the most recent body weight measurement.

Dosing:

for PO administration, animal has either restricted access to food until the next day (12 h) before dosing or are allowed free access to food (depending on whether the PK will be done on fasted or fed animals). In all cases, animals has access to water ad libidum. PO dosing is done by gavage using a curved gavage needle (with rounded tip). All animals are observed for few seconds after injection to make sure there are no side effects. For IV administration, dosing (mice and rat) is done by the tail vein. Compound administration is done by slow (15-60 sec) bolus or by slow IV infusion (up to 1 h) using a calibrated syringe pump. For subcutaneous (dorsal region) and intraperitoneal injections, dosing is done with the appropriate needle and volume as per IACUC guidelines. GPR-405 was administered once by SC injection to each animal in the test group using a hypodermic needle attached to a syringe. No dose was administered to control animals.

Sample Collection:

Sampling is done via the tail, jugular or saphenous vein. Typical time for blood collection for PK are Pre, 5, 15, 30 minutes, 1 h, 2 h, 4 h, 8 h, 24 h. For mice, animals are terminated at each collection (cardiac puncture post-euthanasia). For rats, 200 μl are collected at each time point (max 3 time points per rat in 24 h). Plasma harvested from blood is mixed with 8.5% phosphoric acid (2% v/v of plasma). All plasma samples collected are stored in a freezer, set to maintain −80° C. until bioanalysis. At necropsy, tissues, organs and/or biological fluid are collected, as needed, for analysis. Urine collection is done using metabolic cages when required.

Sample Analysis:

A stock solution of the test article is prepared at 250 μg/mL in DMSO. This solution is quantitatively added to blank mouse or rat plasma to generate a calibration curve. The linear range is from 0.25 to 250 ng/mL. A 175 μL aliquot of plasma is removed from each calibrant tube and processed using solid phase extraction (SPE). Study samples are first diluted with blank control mouse or rat plasma, and 175 μL is aliquoted for processing using SPE. An internal standard working solution (ISWS) is added to each calibrant and study sample (GPR-227 at 100 ng/mL in water). Plasma proteins are precipitated with 175 μL of methanol with 0.5% acetic acid (v/v). After centrifugation at 14,800 RPM for 10 minutes, a 300 μL aliquot of supernatant is diluted in water with 4% phosphoric acid (v/v). The entire diluted sample is extracted by SPE using a Waters Oasis HLB 96-well μElution plate. Samples are washed twice using a solution of acidified water (0.1% TFA, v/v) and 15% MeOH in water (v/v). Purified extracts are eluted into a 96-well collection plate using 40 μL of elution solvent. Standards and study samples are injected onto a Thermo Q-exactive LC-MS/MS system. Chromatographic separation is performed using a reversed-phase C18 HPLC column (Waters CSH C18, 50×2.1 mm, 2.5 μm). Analyte and internal standard are detected using the targeted selected ion monitoring (tSIM) scan mode. Calibration curves are generated by using a weighted (1/concentration) linear regression of the peak area ratios (analyte/internal standard) versus the nominal concentration of the calibration standards. The concentration of the analyte in the study samples was determined by back-calculating the peak area ratios obtained using the linear regression and applying the appropriate dilution factors.

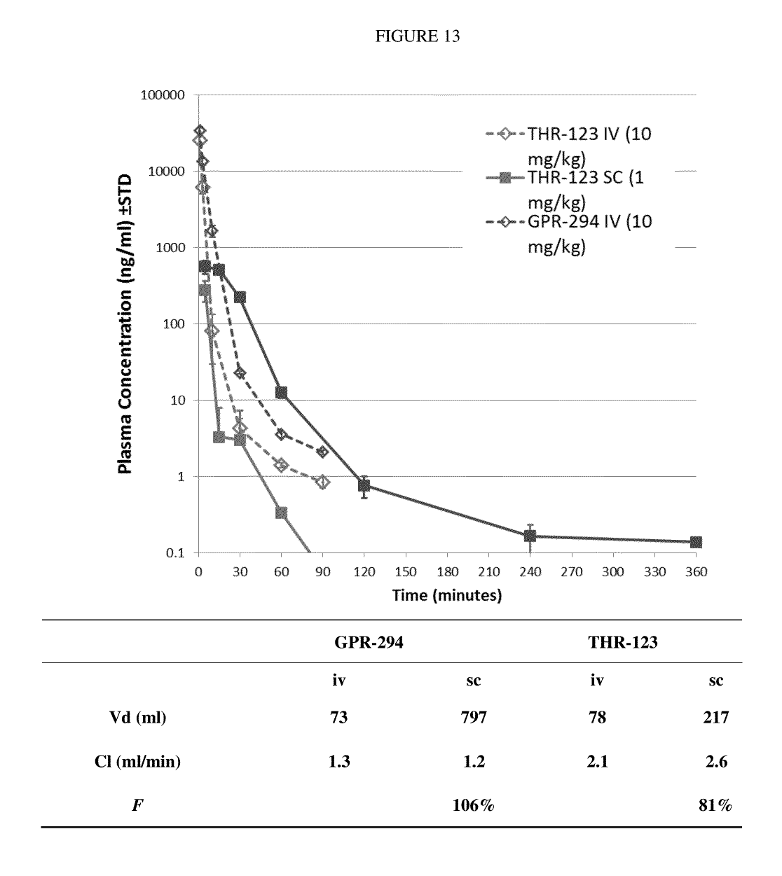

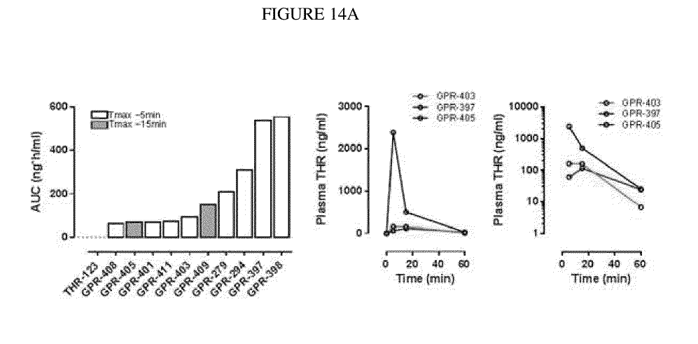

Results of the PK studies for the following Compounds are shown in FIGS. 13 and 14.
THR-123 i.v. and s.c. (Non-stabilized peptide)
GPR-294 i.v. and s.c. (Stabilized peptide)
THR-123, GPR-294(TFA) GPR-294(AcOH), GPR-403, GPR-408, GPR-397, GPR-405, GPR-401, GPR-409, GPR-411, GPR-279, GPR-298 i.v.

As shown in FIG. 13, by sub-cutaneous administration, GPR-294 is 100% bioavailable and THR-123 is 81% bioavailable. The stabilized GPR-294 has a much longer plasma half-life than the non-stabilized THR-123 by either route of administration.

As shown in Table 7, GPR-405 reaches its peak plasma concentration rapidly (<5 min), and then the plasma concentration of GPR-405 decayed in two phases.

TABLE 7
Plasma concentration of GPR-405 over the
PK time-course, as measured by LC-MS/MS

| | | Final | Mean | Standard |
|---|---|---|---|---|
| Time | | Concentration | Concentration | Deviation |
| (min) | Mouse ID | (ng/ml) | (ng/ml) | (ng/ml) |
| 0 | 1001A | 0.0 | 0.0 | 0.0 |
| | 1002A | 0.0 | | |
| 5 | 8013C | 596.4 | 504.1 | 130.5 |
| | 8015C | 411.9 | | |
| | 8014C | 62.4 (outlier) | | |
| 15 | 8001A | 114.2 | 117.0 | 2.4 |
| | 8002A | 118.3 | | |
| | 8003A | 118.4 | | |
| 30 | 8007B | 49.0 | 68.8 | 28.0 |
| | 8008B | 88.6 | | |
| 60 | 8004A | 23.0 | 15.9 | 6.2 |
| | 8005A | 11.9 | | |
| | 8006A | 12.8 | | |
| 240 | 8010B | 0.4 | 0.4 | 0.0 |
| | 8011B | 0.4 | | |

TABLE 8
PK Parameters of GPR-405 given at 1 mg/kg sc in CD1 mice

| Parameter | Unit | Value |
|---|---|---|
| Mean TI Amount | ng | 32500 |
| Dose | ng/kg | 1000000 |
| F | | tbd |
| Cₘₐₓ | ng/ml | 504.1 |
| Tₘₐₓ | min | 5 |
| K′ | min⁻¹ | 0.022 |
| AUC₍₀₋ₜ₎ | ng-min/ml | 8496 |
| AUC_{(0-∞)} | ng-min/ml | 8514 |
| AUMC₍₀₋ₜ₎ | ng*min²/ml | 196073 |
| AUC_{(0-∞)} | ng*min²/ml | 201207 |
| MRT | min | 23.08 |
| K_{eI} | min⁻¹ | 0.04 |
| CL*F | ml/min | 3.83 |
| CL/kg*F | L/min/kg | 0.12 |
| Vd*F | ml | 172.28 |
| Vd/kg*F | L/kg | 5.29 |
| Vss | ml | 90.22 |
| Biological T_{1/2} | (min) | 16.00 |
| Terminal T_{1/2} | (min) | 31.21 |

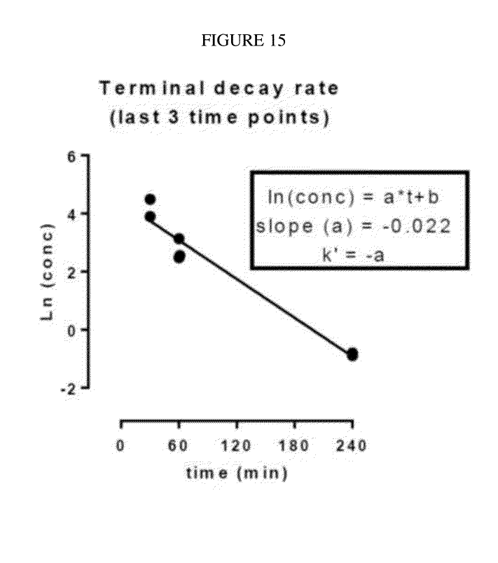

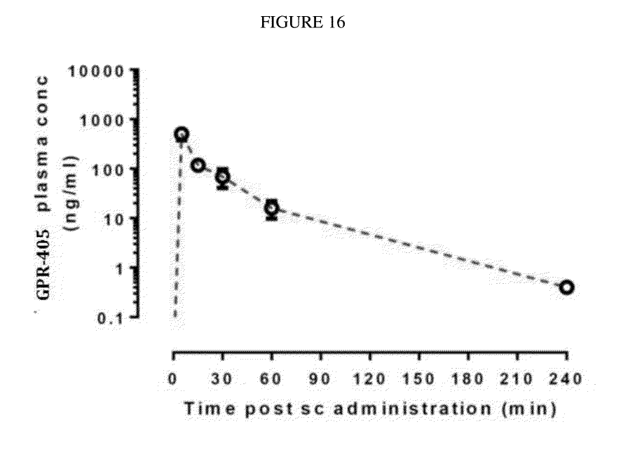

FIGS. 15 and 16 provide the pharmacokinetic profile of GPR-405.TFA following subcutaneous administration of 1 mg/kg in CD1 mice. FIG. 15 provides a log-linear regression fit of the last 3 time points (30, 60, and 240 minutes) that was used to estimate the terminal decay rate (K′). The K′ was determined as the slope of the linear regression. FIG. 16 shows the overall PK profile of GPR-405, with each point representing the mean±SD of the plasma concentration (n=2-3 mice/point). The overall half-life of GPR-405.TFA was 16 minutes approximately and the terminal half-life based on the terminal decay rate was about 30 minutes. The clearance of GPR-405 is about ten times greater than renal clearance (mouse GFR approximately 10 ml/min/kg). GPR-405 has a large volume of distribution, approximately 5 L/kg (mouse blood volume is about 0.1 L/kg), suggesting broad tissue distribution (based on a % bioavailability of 100%). Preliminary data indicates that GPR-405 bioavailability is approximately 60%, decreasing the Vd and Cl values by 40%.

### Example 7

### Adriamycin (ADR) Induced Nephropathy Model

Adriamycine (doxorubicin) is a drug used in chemotherapy. It is known to cause undesirable renal toxicitiy in rat, mainly glomerular damage (via podocyte injury/loss), resulting in proteinuric neophrapthy (Hakroush et al., 2014, J. Am. Soc. Nephrology, 25:927-38), and it is the prototypical model of human primary focal and segmental glomerulosclerosis. ADR-induced mephropathy, in mouse or rat, is a robust in vivo model of proteinuria that can easily and directly be measured in urine, thus allowing for monitoring of disease progression in living animals. It is induced by a single intravenous injection of ADR.

Male CD rats (aged 8 weeks) were acclimatized in the facility for at least 5 days. On Day 0, rats received Adriamycin at a dose level of 7.5 mg/kg by tail vein administration at a dose volume of 5 mL/kg. If needed, an abbocath was used for administration. The abbocath will be flushed with 0.5 mL of 0.9% saline after Adriamycin injection. On selected days (e.g., days 5, 10, 15, 20) animals were put in individual metabolic cages with the normal access of food and water. Urine was collected for ˜18 h (overnight). Urine was brought back to Sponsor on ice for further analysis.

Administration of Vehicle or Test Article:

From Days 0 to last day of study inclusively, animals received the vehicle (0.9% saline or other formulation), test items (e.g. GPR peptides) once daily by intravenous (1.6 ml/kg), subcutaneous (5 ml/kg) or oral [gavage (10 ml/kg) or in drinking water]. The positive control was Enalapril (50 mg/kg po) unless otherwise indicated.

In-Life Procedures:

The following observations and measurements were performed for all animals: Mortality/Morbidity checks once daily by cage-side observation; Body Weight: once pretreatment on Day 0 (prior to dosing) and twice weekly afterwards.

Clinical Pathology:

Once pretreatment and on pre-determined days (e.g. days 5, 10, 15, 20) blood samples (0.7 mL) was collected from the jugular vein in SST tubes and analyzed for serum creatinine and BUN. Once pretreatment and on pre-determined days (e.g. days 5, 10, 15, 20) urine samples were collected overnight from individually housed animals. After collection, serum and urine samples were processed and analyzed for creatinine (serum & urine), BUN (serum) and total protein (urine). Animals usually had free access to food and water during urine collection unless otherwise indicated. The time the animals are placed in urine collection cages and time of sample collection was recorded. Collection was performed overnight for 16 to 18 hours.

Terminal Procedures:

At termination, animals were euthanized by isoflurane inhalation and exsanguination of the abdominal aorta. The right kidney were collected, placed in 10% formalin and kept for future histopathological evaluation. Samples were sometime also placed in modified Karnovsky's fixative for optional electron microscopy (EM) evaluation. One pole of the left kidney will be cut, sectioned in 3 parts and snap frozen in liquid nitrogen and kept at −80° C. for potential genomic, proteomic or biochemical analyses.

### Example 8

### Adriamycin (ADR)-Induced Nephropathy Model: GPR-405

Adult male Sprague-Dawley rats were acclimatized in the facility for at least 7 days. Animals were divided into 3 groups of 6 (test, negative control, positive control). On Day 0, rats received Adriamycin at a dose level of 7.5 mg/kg by tail vein administration at a dose volume of 5 mL/kg. Animals received a single dose of GPR-405, or enalapril or vehicle controls for 21 days post-ADR. Urine and plasma was collected on days 0, 5, 10, 15, and 21 post-ADR. At 21 days, animals were euthanized and kidneys were collected and either frozen or fixed for histology.

Administration of GPR-405, Enalapril and Vehicle:

From Days 0 to 21 inclusively, animals received a daily subcutaneous injection of THR-574.TFA in a single dose of 300 nmol/kg q.d. On the same days, negative control animals received a subcutaneous injection of the vehicle (0.9% saline or other formulation), and positive control animals received Enalapril (50 mg/kg) by oral gavage. The dose volumes were 10 mL/kg for the oral and 5 mL/kg for the subcutaneous injections.

In-Life Procedures:

The following observations and measurements were performed for all animals: Mortality/Morbidity checks once daily by cage-side observation; Body Weight: once pretreatment on Day 0 (prior to dosing) and twice weekly afterwards.

Clinical Pathology:

Once pretreatment and on Day 21, blood samples (0.7 mL) were collected from the jugular vein in SST tubes and analyzed for serum creatinine and BUN. Once pretreatment and on pre-determined days (Days 5, 10, 15, 21) urine samples were collected overnight from individually housed animals. After collection, serum and urine samples were processed and analyzed for creatinine (serum & urine), BUN (serum) and total protein (urine). Animals were fasted overnight for blood and urine sampling, but were not deprived of water during the urine collection procedure. The time the animals are placed in urine collection cages and time of sample collection was recorded. Collection was performed overnight for 16 to 18 hours.

Terminal Procedures:

At termination, animals were euthanized by isoflurane inhalation and exsanguination of the abdominal aorta. One kidney was collected for histopathology. Kidney poles were placed in modified Karnovsky's fixative for optional electron microscopy (EM) evaluation. Sections of 1 mm³ were prepared. The middle section was placed in 10% formalin for possible histopathological evaluation. The other kidney was snap frozen and kept at −80° C.

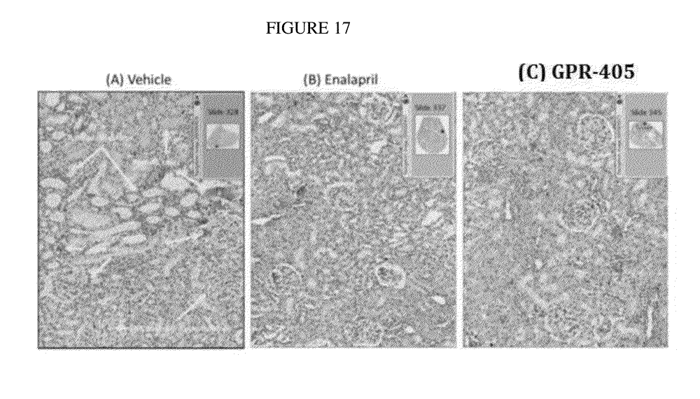

FIG. 17 provides representative microphotographs of H&E-stained 6 μl rate kidney sections (10× magnification) 21 days post ADR administration and treated daily with vehicle, enalapril or GPR-405. ADR-induced lesions are highlighted in yellow in the vehicle group (Panel A), including glomerular hypertrophy, protein casts, tubular dilation and neutrophils (yellow arrows). These renal lesions are absent or reduced in number in enalapril and GPR-405 treated groups.

### Example 9

### Ischemia-Reperfusion Injury Model (IRI)

Surgery:

Male Sprague-Dawley rats (250-300 g) were acclimatized in the facility for at least 5 days. Animals were anesthetized with an isoflurane/O2 mixture, 5% for induction and 1-2% for maintenance of anesthesia. An induction chamber was used and an anesthesia circuit was used during surgery. The abdomen was shaved with clippers and washed with germicidal soap and water, towel dried and swabbed with betadine. The animal was placed on a sterile disposable absorbent towel over a warming pad thermostatically controlled by a rectal thermometer. The animal was monitored continuously for pulse oxometry, respiratory rate and blood pressure. The abdomen was opened using a 3 cm midline incision. Each kidney was isolated and the fat and connective tissue surrounding the renal artery and vein was dissected away using sterile cotton swabs. The right kidney was removed and the renal artery and vein sutured off. Ischemia of the left kidney was initiated by clamping the renal artery and vein for pre-determined time (usually 30-45 minutes) using non-traumatic clamps on each renal pedicle. For sham surgery, the kidneys were isolated as above but not clamped. The incision was covered with sterile saline saturated gauze sponge during the ischemic period. At the conclusion of the ischemic period, the clamps were removed and the kidney was observed to insure rapid re-establishment of blood flow. The animal was rehydrated with 2 ml of sterile saline introduced into the abdominal cavity. The muscle layer was closed with 3-0 silk; the skin was closed with surgical 3-0 silk.

Administration of Vehicle or Test Article:

Typically, in this model, test articles or vehicle were administered by either intravenous bolus or infusion. A jugular intravenous access line was placed for delivery of test article or vehicle. At pre-determined times prior to clamping or post clamp release, the test article or vehicle was administered by i.v. bolus, under anesthesia. For IV, volume was 1.67 ml/kg BW, (e.g., 0.5 ml/300 gram rat over 60 sec). Volumes of working solutions administered were adjusted based on animal body weights.

Blood Sampling and Serum Creatinine Analysis:

Venous blood sample (150 μl) was drawn into BD red top tubes at study initiation for baseline creatinine measurements and at 24 h post-surgery for pathological serum creatinine levels evaluation. Venous blood samples were centrifuged and stored at +4° C. for analysis. Creatinine concentration was measured on a Creatinine Analyzer 2 (Beckman Inc.). The analyzer was standardized with a known standard and the samples were run using a picric acid reaction.

Study Termination:

The study was terminated usually 24 h post-surgery and rats were euthanized by pentobarbital overdose followed by cervical dislocation. Longer time-courses (e.g., 3 days, 5 days) cans be done as needed.

### Example 10

### Adriamycin-Induced Proteinuria in Rat

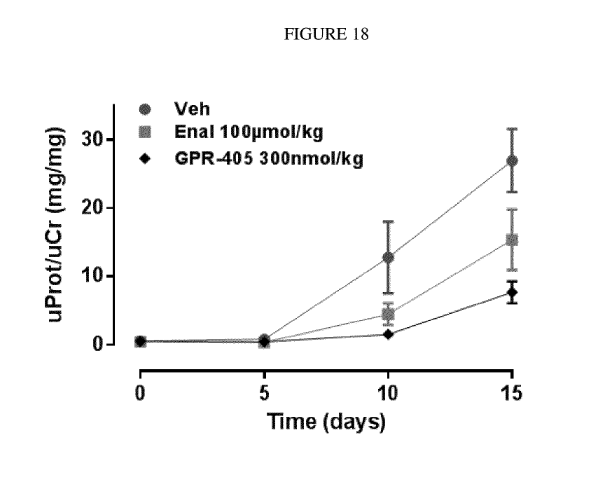

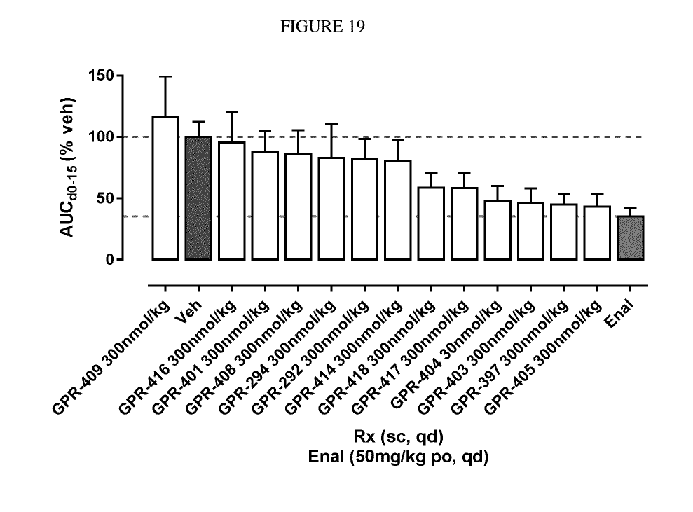

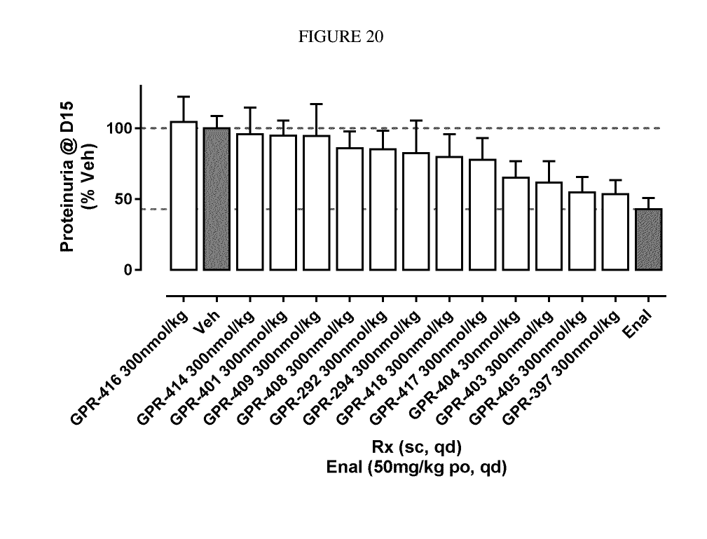

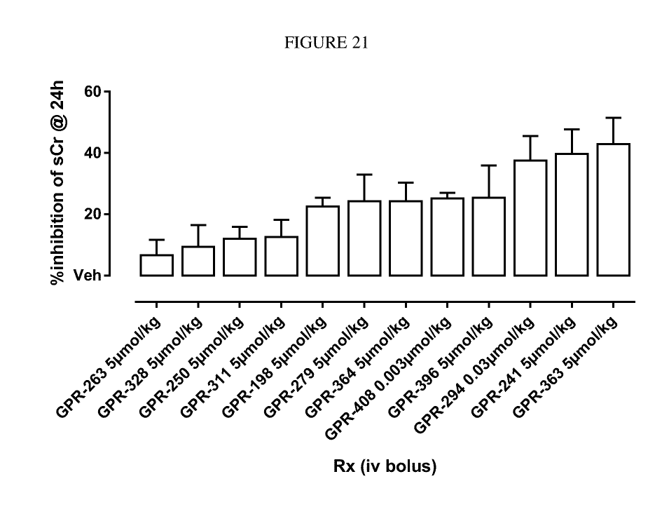

The effect of GPR-405 and enalapril on the time-course of the development of proteinuria was tested (FIGS. 18-21). FIG. 18 shows the development of Adriamycin (ADR)-induced proteinuria over time and the effect of enalapril and GPR-405 (qd). Proteinuria is the ratio urine protein to urine creatinine concentrations (n=6 rat/group). FIG. 19 shows the effect of various Compounds (GPR-401, GPR-409, GPR-285 AcOH, vehicle control, GPR-408, GPR-294 TFA, GPR-292, GPR-403, GPR-397, enalapril and GPR-405) on proteinuria 15 days post-adriamycin treatment. In FIG. 19, the data is pooled from two independent experiments (n=5-12 rats/group). The effect of various Compounds (GPR-409, GPR-285 AcOH, vehicle control, GPR-401, GPR-408, GPR-294 TFA, GPR-292, GPR-403, GPR-397, enalapril and GPR-405) on AUC_{d0-15} is shown in FIG. 20. An AKI-Ischemia-reperfusion injury model was used in the experiments shown in FIG. 21. FIG. 21 shows that various Compounds (GPR-263, GPR-328, GPR-294, GPR-250, GPR-311, THR-123, GPR-279, GPR-364, GPR-396, GPR-241, GPR-363) induce a rise in serum creatinine at 24 hours.

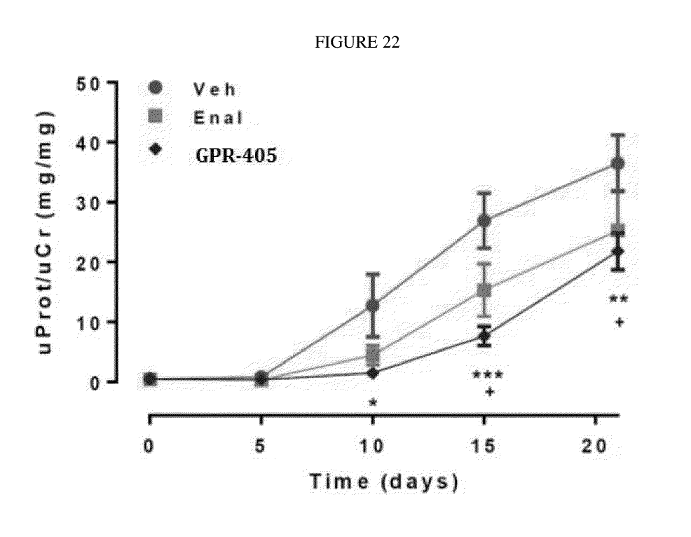

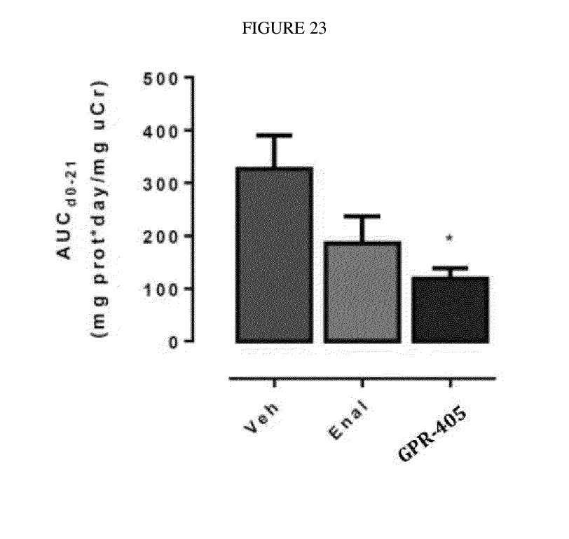

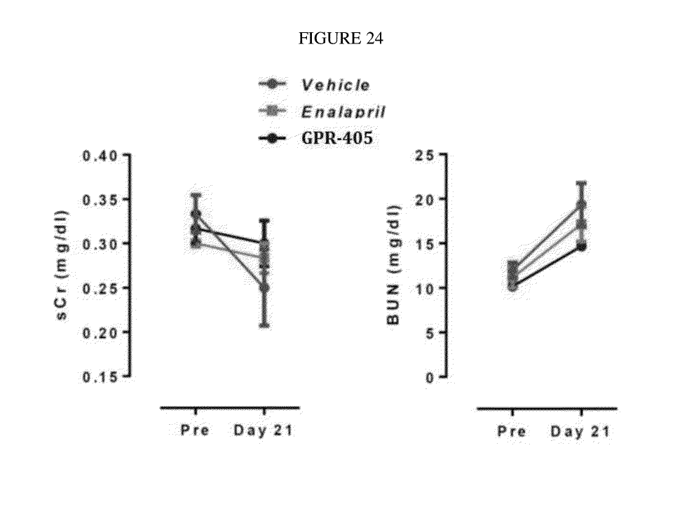

Overall, GPR-405 treatment resulted in a significant inhibition of proteinuria at doses of 300 nmol/kg that persisted up to the end of the 21 day test period. Enalapril at 50 mg/kg was also efficacious. FIG. 22 shows the development of Adriamycin (ADR)-induced proteinuria (casued by a single 7.5 mg/kg dose of Adriamycin) over time and the effects of vehicle, enalapril, or GPR-405 treatment. Proteinuria is the ratio of urine protein to urine creatinine concentrations (n=6 rat/group). FIG. 23 shows the effects of vehicle, enalapril, or GPR-405 treatment on proteinuria between days 0 to 21 following Adriamycin treatment as represented by AUC_{d0-21}. The effect of treatment with enalapril and GPR-405 on changes in serum creatinine (sCr) and blood urea nitrogen (BUN) following Adriamycin treatment is shown in FIG. 24. Neither enalapril nor GPR-405 had any marked effect on sCr or BUN.

### Example 11

### Adriamycin-Induced Gene Expression Changes

Genetic Markers for Kidney Damage:

The efficacy of GPR-405.TFA and enalapril administration in an ADR rat model were tested further by assessing their effects on the expression of genes considered to be markers for kidney damage. The mRNA levels of 19 genes were assayed in dissected kidney cortices of animals treated with GPR-405.TFA, enalapril, or the vehicle. The renal cortex extract from 1 rat that was not administered ADR served as a naïve control and comparator to normalize the data between the groups. The 19 genes assayed were: IL-6, clusterin, cst3, mapk3k7, rdh2, osteopontin, pgarc1a, IL-18, vegfa, nephrin, wt1, IL-1B, kim-1, NGAL, vimentin, hif1a, ID1, synpo, and Mcp1.

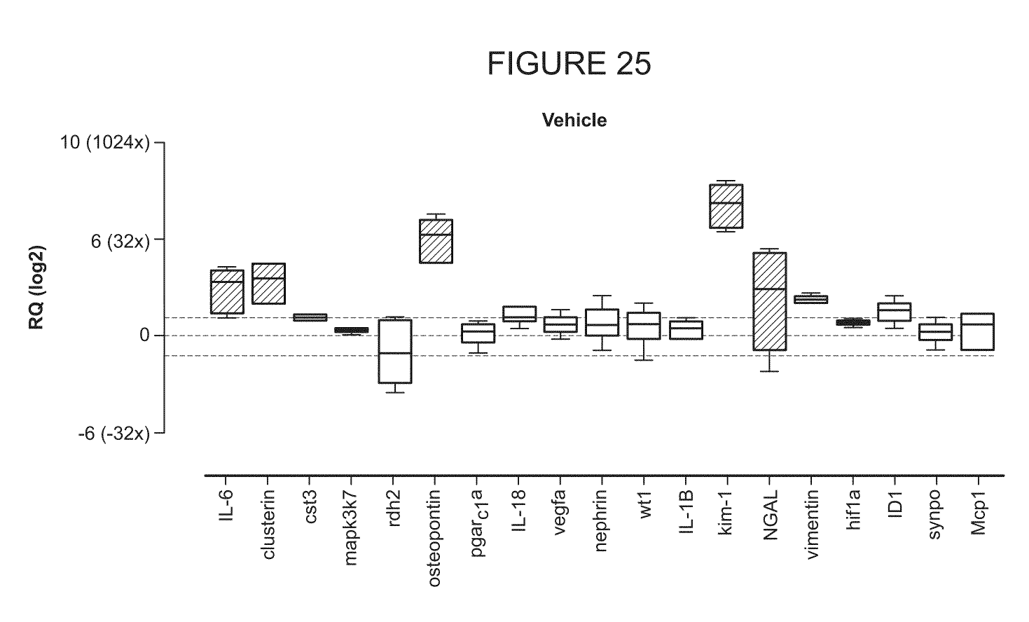

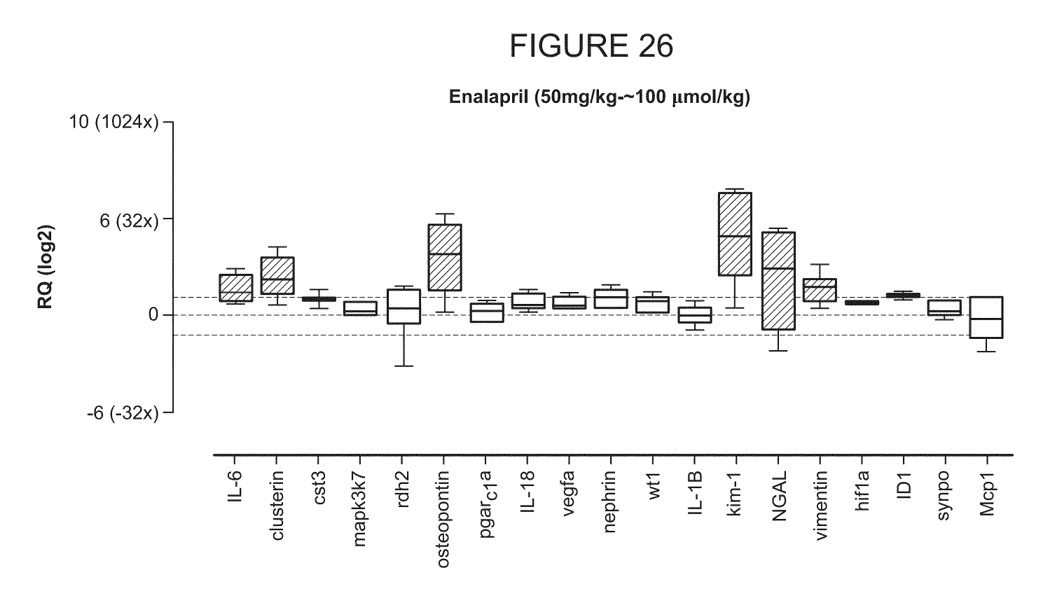

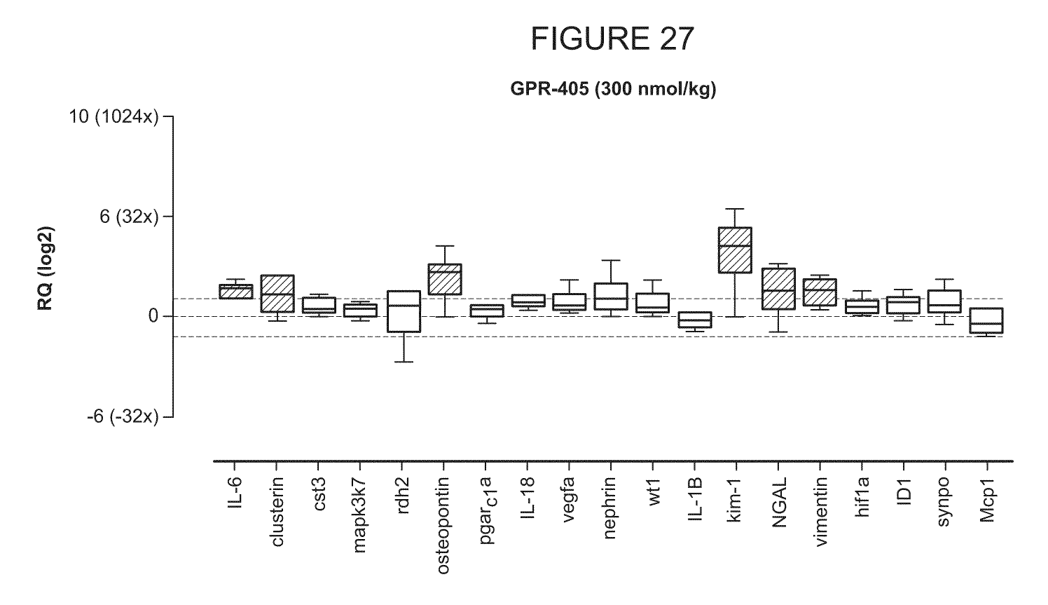

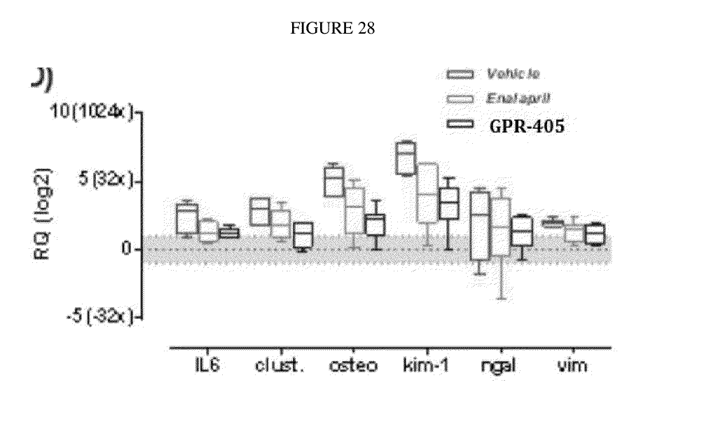

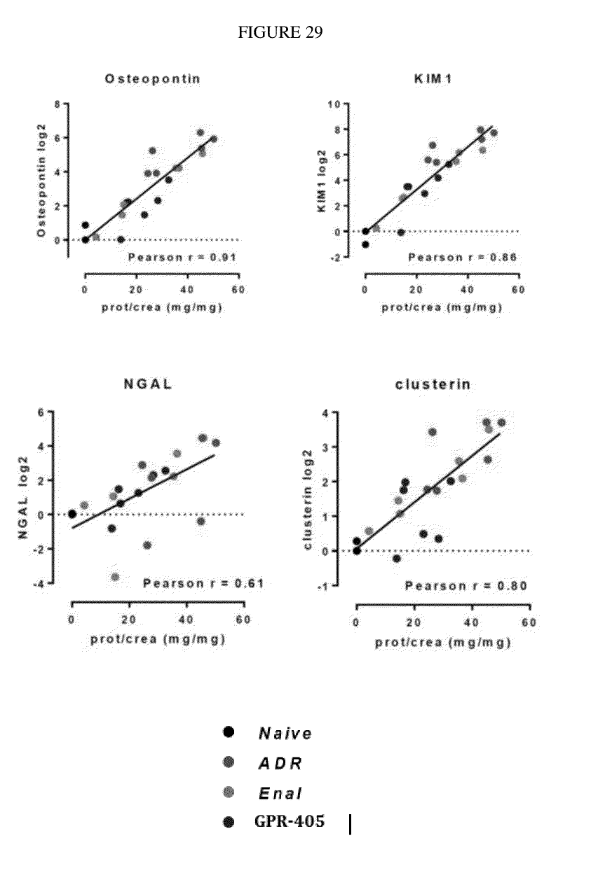

FIGS. 25-28 compare the effects of daily vehicle, enalapril or GPR-405 administration on the mRNA levels of all 19 genes in the dissected kidney cortices of rats 21 days post ADR. RT-PCR on the RNA extracted from the renal cortices of 6 animal per group (vehicle, enalapril and GPR-405) was used to detect changes in mRNA levels (RQ values) for each of the genes. Upon further analysis, significant linear correlation between proteinuria and modulation of KIM1, NGAL, osteopontin and clusterin was observed. Adriamycin treatment increased the transcript levels of 6 genes (IL-6, KIM1, NGAL, clusterin, osteopontin, and vimentin) by greater than 2.5 times, and increased the transcript levels of IL-18 and ID1 by more than 2 times. FIG. 29 illustrates the correlation between proteinuria levels and the expression levels of four genes (Kim1, osteopontin, NGAL, and clusterin) most affected by enalapril and GPR-405 in rats with SDR-induced proteinuria at Day 21. Overall, there was a significant linear correlation between proteinuria and modulation of gene levels of KIM1, NGAL, osteopontin and clusterin.

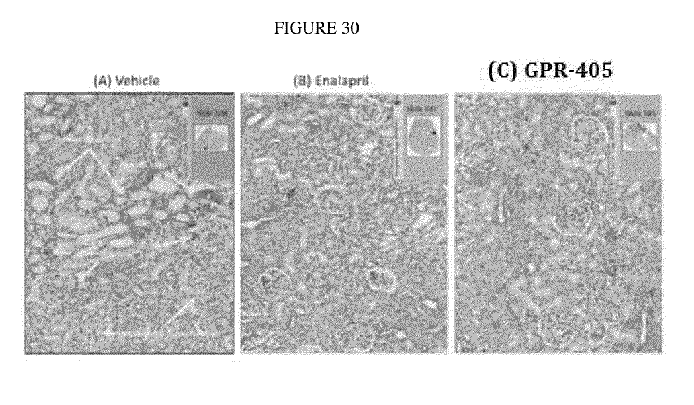

FIG. 30 depicts representative microphotographs of H&E stained 6 μM rat kidney sections (10× magnitude) 21 days post ADR insult treated daily with either vehicle, enalapril or GPR-405. ADR-induced lesions are highlighted in the vehicle group; glomerular hypertrophy, protein casts, tubular dilation, and neutrophils were frequent. Those hallmarks of renal injury were reduced or absent in the enalapril and GPR-405 groups.

### Example 12

### Plasma Stability Studies

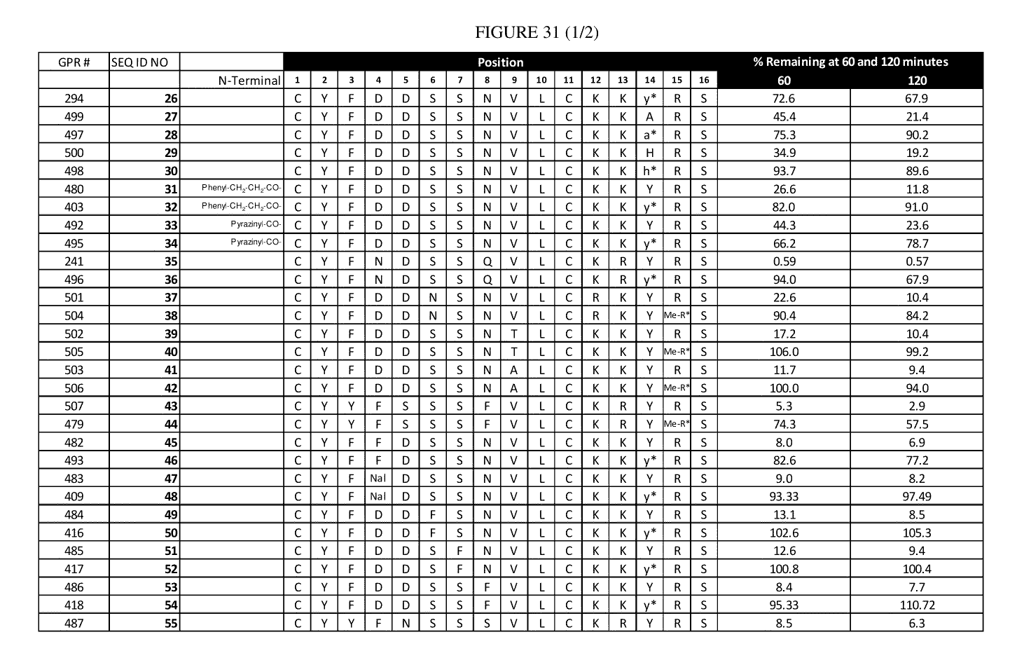

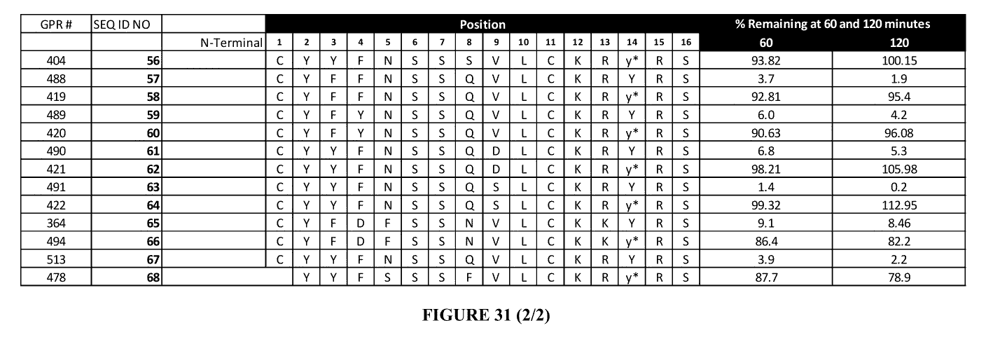

GPR peptides as shown in FIG. 31 (SEQ ID NOs 26-68) were incubated at a concentration of 1 μM in fresh rat plasma over a 2 hour time course. The incubations took place in a water bath set at 37° C. The peptides were added to separate aliquots of plasma for the following time points to be assessed: 0, 5, 15, 30, 60 and 120 minutes. Each time point was assessed in duplicate at a sample volume of 200 μL (196 μL plasma and 4 μL 50 μM peptide standard). Plasma aliquots were pre-incubated at 37° C. for 30 min prior to peptide addition. Zero minute incubated plasma samples were prepared first by protein precipitation (to quench enzymatic activity) prior to peptide addition. Plasma precipitation was performed by addition of 400 μL of cold acetonitrile containing an internal standard (100 nM GPR404 or GPR405) followed with vortexing. Peptide standards were added to all non-zero time course plasma aliquots after the preparation of the 0 min samples and the incubation time course was started. Plasma incubations were stopped at each assigned time course point by protein precipitation. After the time course was completed, the precipitated samples were centrifuged at 2000×g for 15 minutes. A 100 μL aliquot of the resulting supernatant was removed for each sample and transferred to a 96-well plate containing 100 μL 2% trifluoroacetic acid in water. The plate was vortexed and placed in an autosampler for liquid-chromatography and mass spectrometry (LC-MS) analysis. A Waters Acquity UPLC liquid chromatograph coupled with an AB/SCIEX API 4500 mass spectrometer were used for the LC-MS analysis. The chromatographic separation was performed using gradient elution with a reversed-phase C18 UPLC column (Acquity CSH C18 50×2.1 mm, 1.7 μm) equipped with a guard column (VanGuard CSH C18 5×2.1 mm, 1.7 μm). The solvent system consisted of Milli-Q water, acetonitrile and formic acid. Analyte and internal standard were detected using the multiple reaction monitoring (MRM) scan mode. All LC-MS chromatogram peak processing and integration were performed using AB/SCIEX Analyst version 1.6.2 software. Peptide to internal standard peak area ratios were used calculate percentage remaining peptide for each time point. Calculations and data tabulation were performed using Microsoft Excel 2010 software. The results are shown in FIG. 31.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a subject having a disease or disorder, or delaying the progression of a disease or a disorder in a subject, the method comprising administering to the subject an effective amount of a peptide comprising one or more of SEQ ID NO:1-72,
wherein the disease or disorder is a disease or disorder associated with aberrant TDF polypeptide or TDFRP compound target molecule expression or activity,
thereby treating the disorder associated with aberrant TDF polypeptide or TDFRP compound target molecule expression or activity in the subject.

2. The method of claim 1, wherein the disease or disorder is a tissue degenerative disease.

3. The method of claim 2, wherein the tissue degenerative disease is selected from the group consisting of: renal disease, heart disease, traumatic brain injury, stroke, atherosclerosis, arthritis, emphysema, osteoporosis, cardiomyopathy, cirrhosis, degenerative nerve disease, Holt-Oram disease, congenital disease, pulmonary disease, eye disease, diabetic nephropathy, degenerative bone disease, bone disorders, periodontal disease, chronic kidney disease, diabetes, cardiovascular disease, inflammatory disease, immune disease, skeletal disease, reproductive disease, hematopoetic disease, healing disorders, and cancer.

4. The method of claim 1, wherein the disease or disorder is treated by the regeneration of tissues, organs or limbs.

5. The method of claim 4, wherein the tissue is selected from the group consisting of: muscle, bone, skin, epithelial, heart, nerve, endocrine, vessel, cartilage, periodontal, liver, retinal, and connective tissue.

6. A method of treating a subject having a kidney disease or disorder or a method of delaying the progression of a kidney disease or disorder in a subject, comprising administering to the subject an effective amount of a peptide comprising one or more of SEQ ID NO:1-72, thereby treating the kidney disease or disorder, or preventing the progression of the kidney disease or disorder, in the subject.

7. The method of claim 1 or claim 6, wherein the peptide has at least 70% identity to SEQ ID NO:1-72.

8. The method of claim 1 or claim 6, wherein the peptide consists of the amino acid sequence set forth as any one of SEQ ID NOs:1-72.

9. The method of claim 1 or claim 6, wherein the peptide is formulated with a pharmaceutically acceptable carrier.

10. The method of claim 1 or claim 6, wherein the peptide is administered to the subject orally.

11. The method of claim 1 or claim 6, wherein the peptide is administered to the subject topically, enterally, or parenterally.

12. The method of claim 1 or claim 6, wherein the disease or disorder is chronic kidney disease.

13. The method of claim 1 or claim 6, wherein the disease or disorder is a renal dysfunction.

14. The method of claim 13, wherein the renal dysfunction is selected from the group consisting of ureteral obstruction, acute and chronic renal failure, renal fibrosis, and diabetic nephropathy.

15. The method of claim 1 or claim 6, wherein a dosage of 0.0001 to 10,000 mg/kg body weight is administered to the subject per day.

16. The method of claim 1 or claim 6, wherein the administered dosage is from 1 to 100 mg/kg body weight per day.

17. A peptide for treating a disease or disorder associated with aberrant TDF polypeptide or TDFRP compound target molecule expression or activity, wherein the peptide comprises the amino acid sequence set forth as any one of SEQ ID NOs: 1-72.

18. The peptide of claim 17, wherein the peptide has at least 70% identity to SEQ ID NO:1-72.

19. The peptide of claim 17, consisting of the amino acid sequence set forth as any one of SEQ ID NOs:1-72.

20. A pharmaceutical composition comprising the peptide of any one of claims 17-19 and a pharmaceutically acceptable carrier.

21. A kit comprising the peptide of any one of claims 17-19 and instructions for use.

22. A kit comprising the pharmaceutical composition of claim 21 and instructions for use.
